SEQUENCE LISTING (6 sequences)

SEQ ID NO: 1 - DNA, 22 bp, Artificial sequence
  synthesized sequence
ctaaatatcggggagagaccga

SEQ ID NO: 2 - DNA, 29 bp, Artificial sequence
  synthesized sequence
gtacttttaactctctttccaaagtgctt

SEQ ID NO: 3 - DNA, 21 bp, Artificial sequence
  synthesized sequence
ttcatctttccctcacggtac

SEQ ID NO: 4 - DNA, 21 bp, Artificial sequence
  synthesized sequence
acaccattcccccctatctgt

SEQ ID NO: 5 - DNA, 21 bp, Artificial sequence
  synthesized sequence
tgaccaccagcatctgttgaa

SEQ ID NO: 6 - DNA, 19 bp, Artificial sequence
  synthesized sequence
tgccggcgcaaaacacctg

(19) United States
(12) Patent Application Publication
(10) Pub. No.: US 2023/0054926 A1
(43) Pub. Date: Feb. 23, 2023

(54) COMPOSITIONS AND METHODS FOR MICROBIAL TREATMENT OF SKIN DISORDERS

(71) Applicant: DUPONT US HOLDING, LLC, Wilmington, DE (US)

(72) Inventors: AMANDA CHAN, TOWNSEND, DE (US); HELENE FLANNERY, SWEDESBORO, NJ (US); JOHN THOMAS GANNON, HOCKESSIN, DE (US); SEUNG-PYO HONG, HOCKESSIN, DE (US); RAYMOND E JACKSON, NEWARK, DE (US); JONATHAN DAVID PETERSON, EGG HARBOR TWP, NJ (US); RICK W YE, WILMINGTON, DE (US)

(21) Appl. No.: 17/796,433

(22) PCT Filed: Jan. 29, 2021
(86) PCT No.: PCT/US2021/015670

§ 371 Date: Jul. 29, 2022

Related U.S. Application Data
(60) Provisional application No. 63/116,193, filed on Nov. 20, 2020.
(60) Provisional application No. 62/968,228, filed on Jan. 31, 2020.

(51) Int. Cl. A61K 36/064; A61P 31/10

(52) U.S. Cl. CPC A61K 36/064; A61P 31/10

(57) ABSTRACT

The present disclosure is directed towards skin care compositions, skin care formulations, and methods for providing treatment of scalp disorders. More specifically, the present disclosure is directed towards methods and compositions comprising at least one microorganism of the genus Yarrowia and/or a fraction thereof, and/or a cell lysate thereof, and/or fermentate thereof, and/or metabolite thereof, for treating a scalp disorder, including dandruff.

### CROSS REFERENCE

### Field of the Disclosure

The present disclosure is directed towards skin care compositions, skin care formulations, and methods for providing treatment of scalp disorders. More specifically, the present disclosure is directed towards methods and compositions comprising at least one microorganism of the genus Yarrowia and/or a fraction thereof, and/or a cell lysate thereof, and/or fermentate thereof, and/or metabolite thereof for treating a scalp disorder, including dandruff.

### REFERENCE TO SEQUENCE LISTING SUBMITTED ELECTRONICALLY

The content of the sequence listing electronically submitted with the application as an ASCII text file (20210127_NB41714PCT_ST25; Size: 2 KB; Created: Jan. 27, 2021) forms part of the application and is hereby incorporated herein by reference in its entirety.

### BACKGROUND

The skin functions as a barrier protecting the organism from drying out as well as protecting the organism against the penetration of external, often harmful, substances.

The skin is also home to a diverse population of microbes, the majority of which are commensal (nonpathogenic permanent residents) or transient (temporary residents) organisms. In pathogenic interactions, only the microbe benefits, while the host is eventually harmed. Many skin pathogens can be typically found living on the skin as commensal organisms, but microbial dysbiosis (or microbial imbalance), host genetic variation, and immune status may drive the transition from commensal to pathogen (Findley, K. and Grice, E. A., The Skin Microbiome: A Focus on Pathogens and Their Association with Skin Disease, PLoS Pathog. 2014, 10).

The epidermis constitutes the outermost region of the skin tissue and as such forms the actual protective sheath against the environment. The outer layer of the epidermis (Stratum corneum or Horny layer) is the part which is in contact with the environment and the particular structure of the horny layer protects the skin as well as stabilizes its own flexibility by binding a defined amount of water (P. M. Elias, Structure and Function of the Stratum Corneum Permeability Barrier, Drug Dev. Res. 13, 1988, 97-105).

Spatially; the skin microbiota may extend to subepidermal compartments (Nakatsuji, T et al., The Microbiome Extends to Subepidermal Compartments of Normal Skin. Nat. Commun. 2013, 4). Regions such as the face, chest, and back, areas with a high density of sebaceous glands, promote growth of lipophilic microorganisms such as Propionibacterium and Malassezia.

Malassezia is a predominant fungus of the skin microbiota and found on virtually everybody's scalp and implicated in the most common skin disorders such as, but not limiting to, seborrheic dermatitis, dandruff, and tinea versicolor. Dandruff is the common term for seborrhea of the scalp. It is mainly associated with Malassezia restricta (M. restricta) and Malassezia globosa (M. globosa) and has a very high prevalence of nearly 50% of the population (Schommer, N. N.; Gallo, R. L., Structure and Function of the Human Skin Microbiome. Trends Microbiol. 2013, 21, 660-668). Improvements in the disease can be achieved by therapeutic application of antifungal, but not antibacterial agents. The mechanisms underlying pathogenicity are incompletely understood. Impaired skin barrier function facilitates the course of the disease (Harding, C. R et al., Dandruff: a condition characterized by decreased levels of intercellular lipids in scalp stratum corneum and impaired barrier function. Arch. Dermatol. Res. 2002, 294, 221-230).

Malassezia species (Malassezia spp) do not have fatty acid synthase, so they have to rely on sebum lipids for carbon source. They also lack delta 2,3-enoyl-CoA isomerase for efficient unsaturated FA (e.g. oleate) utilization. Malassezia species feeds on sebum fat (by secreting a lipase or lipases that splits triglycerides into irritant fatty acids), and as sebum fat is broken down, free fatty acids (such as oleic acid) are released as by-product. Many people are sensitive to free fatty acids as they can induce hyperproliferation and scaling, or induce the release of arachidonic acid, which is also involved in inflammation, and their scalp responds by becoming irritated. In respond to the irritation, the scalp starts to become inflamed, red, and itchy, and the body shed skin cells faster than usual, in attempt to shed the irritant. The shedding of skin causes visible flakes to appear on the scalp, which is dandruff.

There remains a need to find methods and skin care compositions for providing treatment of scalp disorders, such as but not limiting to methods and compositions for the treatment of a dandruff condition of the scalp.

### SUMMARY

The present disclosure is directed to compositions and methods for providing treatment of scalp disorders. More specifically, the present disclosure is directed towards methods and compositions comprising at least one microorganism of the genus Yarrowia and/or a fraction thereof, and/or a cell lysate thereof, and/or fermentate thereof, and/or metabolite thereof for treating a scalp disorder, including dandruff.

Unexpectedly, the inventors have observed that a microorganism, in particular of the genus Yarrowia, can consume free fatty acids generated by lipid degrading activities of the dandruff inducing Malassezia species, and M. globosa in particular, thereby making it possible to reduce dandruff conditions in subjects in need thereof. Furthermore, the inventors have unexpectedly observed that a microorganism of the genus Yarrowia and/or a fraction thereof, and/or a cell lysate thereof, and/or fermentate thereof, and/or metabolite thereof, can reduce the growth of Malassezia species, removes biofilm of Malassezia species and prevent or reduce biofilm formation of Malassezia species.

In one embodiment, the composition is a skin care composition for use in the treatment of a scalp disorder, comprising an effective amount of at least one microorganism of the genus Yarrowia and/or a fraction thereof, and/or a cell lysate thereof, and/or fermentate thereof, and/or metabolite thereof, wherein said composition reduces and/or treats said scalp disorder.

In one aspect the scalp disorder is selected from the group consisting of a dandruff condition of the scalp (seborrheic dermatitis), unbalanced ecoflora of the scalp, discomfort of the scalp, tinea versicolor, dry skin, irritated skin, or any one combination thereof.

In one embodiment, the composition is a skin care composition for use in the treatment of a scalp disorder, comprising an effective amount of at least one microorganism of the genus Yarrowia and/or a fraction thereof, and/or a cell lysate thereof, and/or fermentate thereof, and/or metabolite thereof, wherein said composition reduces and/or treats said scalp disorder, wherein the composition degrades lipids selected from the group consisting of sapienic acid (C16:1 cis-6), palmitic acid (C16:0), myristic acid (C14:0), petroselinic acid (C18:1 cis-6), pentadecylic acid (C15:0), stearic acid (C18:0), lauric acid (C12:0), leic acid, and any one combination thereof.

In one embodiment, the composition is a skin care composition for use in the treatment of a scalp disorder, comprising an effective amount of at least one microorganism of the genus Yarrowia and/or a fraction thereof, and/or a cell lysate thereof, and/or fermentate thereof, and/or metabolite thereof, wherein said composition reduces and/or treats said scalp disorder, wherein the composition reduces the growth of Malassezia species.

In one embodiment, the composition is a skin care composition for use in the treatment of a scalp disorder, comprising an effective amount of at least one microorganism of the genus Yarrowia and/or a fraction thereof, and/or a cell lysate thereof, and/or fermentate thereof, and/or metabolite thereof, wherein said composition reduces and/or treats said scalp disorder, wherein the composition removes biofilm of Malassezia species.

In one embodiment, the composition is a skin care composition for use in the treatment of a scalp disorder, comprising an effective amount of at least one microorganism of the genus Yarrowia and/or a fraction thereof, and/or a cell lysate thereof, and/or fermentate thereof, and/or metabolite thereof, wherein said composition reduces and/or treats said scalp disorder, wherein the composition prevents or reduces biofilm formation of Malassezia species.

In one embodiment, the composition is a skin care composition for use in the treatment of a scalp disorder, comprising an effective amount of at least one microorganism of the genus Yarrowia and/or a fraction thereof, and/or a cell lysate thereof, and/or fermentate thereof, and/or metabolite thereof selected from the group consisting of Yarrowia lipolytica ATCC 20362, Yarrowia lipolytica ATCC 9773, Yarrowia lipolytica ATCC 18942, Yarrowia lipolytica ATCC 20177, Yarrowia lipolytica CBS2073, Yarrowia lipolytica Phaff #50-47, or any one combination thereof.

In one embodiment, the composition is a skin care product comprising an effective amount of a skin care composition for use in the treatment of a scalp disorder, comprising an effective amount of at least one microorganism of the genus Yarrowia and/or a fraction thereof, and/or a cell lysate thereof, and/or fermentate thereof, and/or metabolite thereof, wherein said composition reduces and/or treats said scalp disorder, and one or more dermatologically or skin care acceptable component.

In one embodiment, the composition is a skin care product selected from the group consisting of a lotion, a serum, a jelly, a cream, a gel, an emulsion, a mask, a patch, or a stick comprising one or more dermatologically or skin care acceptable components and at least about 1%, 2%, 3%, 4%, 5%, 6%, 7%, 8%, 9% up to 10% of the skin care formulation described herein on a weight basis relative to a total weight of said skin care formulation.

In one embodiment, the method is a method for treating a scalp disorder in a subject in need thereof, comprising administering an effective amount of at least one microorganism of the genus Yarrowia and/or a fraction thereof, and/or a cell lysate thereof, and/or fermentate thereof, and/or metabolite thereof, to said subject.

In one embodiment, the method is a method for treating and/or reducing a dandruff condition of the scalp in a subject in need thereof, comprising administering an effective amount of at least one microorganism of the genus Yarrowia and/or a fraction thereof, and/or a cell lysate thereof, and/or fermentate thereof, and/or metabolite thereof, to said subject.

In one embodiment, the method is a method for treating and/or reducing a dandruff condition of the scalp in a subject in need thereof, comprising topically administering a skin care product comprising a skin care composition described herein to said subject.

Further provided herein is a kit, comprising the compositions described herein and instructions for the use thereof to treat a skin condition. In some embodiments, the kit further comprises one or more applicator configured to apply the composition.

### DETAILED DESCRIPTION

The features and advantages of the present disclosure will be more readily understood, by those of ordinary skill in the art from reading the following detailed description. It is to be appreciated that certain features of the disclosure, which are, for clarity, described above and below in the context of separate embodiments, may also be provided in combination in a single element. Conversely, various features of the disclosure that are, for brevity, described in the context of a single embodiment, may also be provided separately or in any sub-combination. It will be understood that in the following, embodiments referred to in relation to one broad aspect of the invention are equally applicable to each of the other broad aspects of the present invention described above. It will be further understood that, unless the context dictates otherwise, the embodiments described below may be combined.

### Scalp Disorders

For the purpose of the present disclosure; the term “scalp disorder” includes a dandruff condition of the scalp (seborrheic dermatitis), unbalanced ecoflora of the scalp, discomfort of the scalp, tinea versicolor, dry skin, irritated skin, or any one combination thereof.

For the purpose of the present disclosure, the term “dandruff condition” refers to a condition manifested by a scalp presenting excessive dryness or excessive secretion of sebum, which, depending on the case, may be characterized by the presence of dry or greasy or oily dandruff, or even pruritic and/or an inflammation of the epidermis.

Dry dandruff conditions reflect a xerosis of the scalp, which may be combined with excessively rapid renewal of its stratum corneum. Dry dandruff flakes are generally in the form of small white or grey flakes and are spread over the scalp and on the clothing, giving rise to an unaesthetic visual effect.

The itching associated with dryness of the scalp may lead to erythema, pruritus or even inflammation.

Greasy or oily dandruff conditions are one of the forms of seborrhoeic dermatitis. Individuals suffering from seborrhoeic dermatitis have an erythematous scalp covered with large, greasy or oily, yellow scales which accumulate so as to form packets. They have a pruritic scalp, and often have burning sensations on the affected areas. These phenomena may be amplified by the presence of pathogenic microorganisms, especially Malassezia species (Malassezia spp.). Malassezia species described herein include, but are not limited to, Malassezia restricta (M. restricta) and Malassezia globosa (M. globosa). These microorganisms having the property of releasing fatty acids from the sebum may impair the barrier function of the epidermis and give rise to inflammation.

During dandruff conditions of the scalp, the cutaneous barrier is unbalanced, its integrity and its hydration are impaired, and its ecoflora is disturbed. The skin of the scalp is irritated and pruritic, brittle, less hydrated, and sensitive to infections.

Unexpectedly, the inventors have observed that a microorganism, in particular of the genus Yarrowia, can consume free fatty acids produced by lipid degrading activities of the dandruff inducing Malassezia species. The use of a microorganism, in particular of Yarrowia, in accordance with the disclosure can result in the reduction of free fatty acids produced by lipid degrading activities of the dandruff inducing Malassezia species, and M. globosa in particular, thereby making it possible to reduce dandruff conditions in subjects in need thereof. Fatty acids might be produced from other lipophilic bacteria. The reduction in occurrence of fatty acids can reduce dandruff and other skin disorders caused by free fatty acids

This decrease can be reflected by a reduction in the phases of scratching the scalp and a reduction in the impairment of the barrier function resulting therefrom. The skin is then less irritated and less pruritic, and the presence of the dandruff is reduced, or even eliminated.

### Microorganisms, Fractions Thereof, Cell-Lysates Thereof, Fermentates Thereof and Metabolites Thereof

Surprisingly and unexpectedly, the results as described herein showed that when Y. lipolytica and M. globosa were grown together, growth of M. globosa in presence of Y. lipolytica was greatly reduced (e.g. Y. lipolytica is inhibiting Malassezia sp.), and as such indicated that microorganisms of the genus Yarrowia can be used as a microbial treatment for a dandruff condition. As described herein (Table 2, Example 3), M. globosa expressed lipase activity in the cell free medium (indicating that the lipase was secreted), but Y. lipolytica did not show significant lipase activities in the cell free supernatant, presumably because Yarrowia's lipase activities were mostly cell-bound or cell-associated. This is a surprising and favorable feature for a microbial treatment because the lipase activities would not be left on the skin and cause free fatty acid accumulation after microbial treatment is completed. As described herein, oleic free fatty acids (FFA) generated by lipase activity of M. globosa were efficiently consumed by Y. lipolytica but not by M. globosa (Example 5, Table 4), which is also in agreement with the result (Example 2, Table 1) showing that M. globosa was unable to utilize (and thus unable or weak growth on) oleic acid, whereas oleic acids were efficiently assimilated by Yarrowia lipolytica. Since oleic free fatty acid (FFA) is considered proinflammatory, efficient removal of this FFA is an important attribute of the Yarrowia microbial treatment against dandruff or other skin disorders caused by proinflammatory oleic FAA.

Furthermore, the inventors have unexpectedly observed that a microorganism of the genus Yarrowia and/or a fraction thereof, and/or a cell lysate thereof, and/or fermentate thereof, and/or metabolite thereof, can reduce the growth of Malassezia species, removes biofilm of Malassezia species and prevent or reduce the biofilm formation of Malassezia species.

As used herein, “microorganism” or “microbe” refers to a bacterium, a fungus, a virus, a protozoan, and other microbes or microscopic organisms.

In some embodiments, the at least one microorganism of the genus Yarrowia can be subjected to treatments that render them non-replicating, for example, exposure to heat, desiccation, y-irradiation, or UV-irradiation. A non-replicating Yarrowia can be a dead cell or a living cell that has been rendered incapable of cell division. A non-replicating Yarrowia can be an intact cell or a cell that has undergone partial or complete lysis. In some embodiments, the non-replicating cells can include a mixture of intact and lysed cells.

As used herein, the term “probiotic” or “probiotic microorganism” are used interchangeably herein and refer to a live microorganism (including bacteria or yeasts for example) which, when administered (topically or orally) in sufficient amounts, beneficially affects the host organism, i.e. by conferring one or more demonstrable benefits, such as a reduced dandruff condition, on the host organism.

In one aspect the microorganism suitable for use in the present invention includes the microorganism of the genus Yarrowia.

In one embodiment, the at least one microorganism of the genus Yarrowia is at least one microorganism selected from the group consisting of Yarrowia lipolytica ATCC 20362, Yarrowia lipolytica ATCC 9773, Yarrowia lipolytica ATCC 18942, Yarrowia lipolytica ATCC 20177, Yarrowia lipolytica CBS2073, Yarrowia lipolytica Phaff #50-47, or any one combination thereof. As used herein, the term “fraction” or “fraction of the at least one microorganism of the genus Yarrowia” or “fraction of the at least one microorganism of Yarrowia” or “fraction thereof” more particularly denotes a fragment of the said microorganism, which has efficacy in the treatment of dandruff conditions of the scalp by analogy with the said whole microorganism. A fraction of the at least one microorganism of the genus Yarrowia includes metabolites (also referred to as Yarrowia metabolites) obtained from said at least one microorganism of the genus Yarrowia. In one aspect the fraction of the at least one microorganism of the genus Yarrowia comprises one or more metabolite(s) (active compound(s) derived from the metabolism of a Yarrowia microorganism and also having efficacy in the treatment of a scalp disorder.

As used herein, the term “metabolite(s)” or “metabolite(s) of the at least one microorganism of the genus Yarrowia” or “metabolite(s) thereof” or “Yarrowia metabolite(s)” or “metabolite actives” are used interchangeably and refer to any substance derived from the metabolism of a Yarrowia microorganism and also having efficacy in the treatment of a scalp disorder. In one aspect, the one or more metabolite(s) were produced during the culture (fermentation) of the least one microorganism of the genus Yarrowia for use in the treatment of a scalp disorder. These metabolites of the at least one microorganism of the genus Yarrowia for use in the treatment of a scalp disorder include, but are not limited to, primary metabolites (metabolites directly involved in normal growth, development and reproduction), soluble metabolites, peptides, proteins, nucleotides, secondary metabolites, polynucleotides and polysaccharides.

It will be apparent that the fraction may be used directly in the formulations of the present invention, or that one or more of the actives (metabolites) may be isolated form the fraction by any suitable means prior to use.

According to one embodiment, the Yarrowia metabolites and/or fractions that are suitable for use in the invention may be administered in the form of a lysate.

As used herein, the term “cell lysate” or “lysate” refers to cells which have been lysed by any suitable means. The term “cell lysate” or “lysate” conventionally denotes a material obtained after the destruction or dissolution of biological cells via a phenomenon known as cell lysis, thus giving rise to the release of the intracellular biological constituents naturally contained in the cells of the microorganism under consideration. For the purposes of the present disclosure, the term “lysate” is used without preference to denote the whole lysate obtained via lysis of the microorganism under consideration or only a fraction thereof. The lysate used is thus totally or partially formed from the intracellular biological constituents and from the constituents of the cell walls and membranes. Advantageously, a lysate used for the invention may be the whole lysate obtained via lysis of the microorganism under consideration. This cell lysis may be accomplished by any suitable means, such as but not limiting to, an osmotic shock, a heat shock, ultrasonication, sonication, homogenization, shearing, chemical lysis or under a mechanical stress of centrifugation type.

In one aspect the cell lysate of the at least one microorganism of the genus Yarrowia comprises one or more metabolite(s) (active compounds) derived from the metabolism of a Yarrowia microorganism and also having efficacy in the treatment of a scalp disorder.

It will be apparent that the cell lysate may be used directly in the formulations of the present invention, of that one or more of the actives (metabolites) may be isolated form the cell lysate by any suitable means prior to use.

A lysate may be used in various forms, in the form of a solution or in a pulverulent form. The microorganism(s) may be included in a composition according to the invention in live, semi-active or inactivated or dead form.

For the purposes of the invention, an “inactivated” or “dead” microorganism is a microorganism that is no longer capable of forming colonies in cultures. The dead or inactivated microorganisms may have intact or broken cell membranes. The dead or inactivated microorganisms may be obtained via any method known to those skilled in the art.

In some embodiments, the cell debris is removed prior to use. In one embodiment the cell lysates are filtered prior to use. In one embodiment, the cells are lysed by, for example sonication, homogenization, shearing or chemical lysis.

As used herein, the term “fermentate” is to be understood as a composition for which one or more living microbial strains have been propagated in a nutrient medium. In one aspect, “fermentate” refers to the supernatant of a cell culture of at least one microorganism of the genus Yarrowia from which the cells have been removed. In one embodiment the cells are removed by centrifugation. In one embodiment the fermentate (supernatant of a cell culture) is obtained by filtration of the culture medium in which Yarrowia cells were cultivated.

In one aspect the fermentate of the at least one microorganism of the genus Yarrowia comprises one or more metabolite(s) (active compounds) derived from the metabolism of a Yarrowia microorganism and also having efficacy in the treatment of a scalp disorder.

It will be apparent that the fermentate may be used directly in the formulations of the present invention, or that one or more of the actives (metabolites) may be isolated form the fermentate by any suitable means prior to use.

In one aspect the fermentate may comprise one or more metabolites, such as but not limiting to soluble metabolites, that were produced during the fermentation of at least one microorganism of the genus Yarrowia.

In one embodiment a fermentate originating from the culture (fermentation) of at least one microorganism of the genus Yarrowia may be used in the methods and/or uses of the present invention.

The nutrient medium used for preparing the fermentate is any medium comprising necessary nutrients suitable for propagating selected microorganisms. Suitable nutrients include but are not limited to amino peptides, peptides, yeast extract and/or vitamins. The medium can be based on dairy products, such as milk, cereals, fruits and/or vegetables.

As used herein, the term “soluble metabolite” refers to a metabolite or metabolites present in the supernatant of a cell culture from which the cells have been removed. In one embodiment the culture is grown to a cell density of at least about OD600 0.5. In one embodiment the cells are removed by centrifugation. In one embodiment the supernatant is filtered. It will be apparent that the supernatant may be used directly in the formulations of the present invention, or that one or more of the metabolites may be isolated form the supernatant by any suitable means prior to use.

In some embodiments, the compositions of the invention can include Yarrowia fermentates, from which all or substantially all, of the Yarrowia cells have been removed, Methods for separating cells from growth media are well known in the art and can rely upon physical methods, for example, centrifugation to produce a cell pellet and a culture supernatant, filtration, ultrafiltration, tangential flow-filtration, normal flow filtration or reverse osmosis. Alternatively, or in addition, the separation method can be ligand-based and include, for example, an antibody that specifically binds to Yarrowia. The antibody can be coupled to a solid support such as a magnetic bead.

In some embodiments, the compositions of the invention include Yarrowia that are partially or substantially isolated from the media in which they were grown. The Yarrowia can be live or non-replicating, e.g., inactivated, for example, by heat-treatment. The cells can be lyophilized or freeze-dried under conditions that preserve cell viability. Methods of lyophilizing are well known in the art.

In one embodiment the fermentate may comprise Yarrowia consisting essentially of nonviable cells (e.g. intact cells).

In another embodiment the fermentate may comprise Yarrowia consisting essentially of viable cells (e.g. intact non-cultivatable cells).

The term “consisting essentially of” in the context of the fermentate includes that at least 90% of Yarrowia have the indicated property (e.g. intact non-viable cells) or viable cells (e.g. intact non-cultivatable cells). Suitably at least 95% have the indicated property. Suitably at least 97% have the indicated property. Suitably at least 99% have the indicated property. In some embodiments at least 100% have the indicated property.

A “cell-free fermentate” (synonymous to the term “fermentation supernatant”) as used herein means that the fermentate is substantially free of viable Yarrowia cells.

In one embodiment, the composition is a skin care composition for use in the treatment of a scalp disorder, comprising an effective amount of a fermentation supernatant from a fermentation of at least one microorganism of the genus Yarrowia, wherein said composition reduces and/or treats said scalp disorder.

In one embodiment, the fermentate for use in the compositions and methods and/or uses of the present invention may be substantially free of viable Yarrowia cells, typically containing zero (or substantially) viable cells/mL fermentate.

### Skin Care Compositions and Skin Care Products for the Treatment of Skin Disorders Such as a Dandruff Condition.

As used herein the term “skin care composition” refers to a composition comprising at least one skin care benefit agent capable of providing a skin care benefit.

As used herein the term “skin care benefit agent” or “active agent” are used interchangeably, and refer to a microorganism of the genus Yarrowia and/or a fraction of said microorganism, and/or a cell lysate of said microorganism, and/or a fermentate of said microorganism, and/or a metabolite of said microorganism that can provide a skin care benefit.

As used herein the term “skin care benefit” refers to a benefit provided by an active agent (or skin care composition and/or skin care product comprising an effective amount of said active agent) when applied topically to a skin. In one aspect of the invention the skin care benefit is selected from the group consisting of preventing a dandruff condition, reducing a dandruff condition, treatment of a dandruff condition, reducing the occurrence of Malassezia species on the skin (scalp), removing biofilm formation of Malassezia species on the skin (scalp), preventing or reducing biofilm formation of Malassezia species on the skin (scalp), improving the barrier function of the skin, skin moisturizing (protecting the skin against dehydration by maintaining, restoring and/or strengthening the moisturization of the skin) or any one combination thereof.

As used herein, the term “biofilm” refers to a community of microorganisms embedded in an extracellular polymer matrix attached to a surface. The extracellular polymer matrix is a polymeric conglomeration generally composed of extracellular DNA, proteins, and polysaccharides. A biofilm may have one or more microorganisms and further includes water and may include other trapped particles. The microorganisms may be gram positive or gram-negative bacteria (aerobic or anaerobic); algae, protozoa, and/or yeast or filamentous fungi. In one embodiment, the biofilm is living cells including one or more Malassezia species.

As used herein, “surface” means any structure having sufficient mass to allow for attachment of biofilm. A surface includes a hard surface and a soft surface. Hard surfaces include, but are not limited to metal, glass, ceramics, wood, minerals (rock, stone, marble, granite), aggregate materials such as concrete, plastics, composite materials, hard rubber materials, and gypsum. Other surfaces may be biological surfaces, such as skin, scalp, or keratin.

Additional benefit agents for skin care can include antidandruff active agents.

Examples of antidandruff active agents include keratolytic agents such as salicylic acid and sulphur in its various forms, regulators of keratinization such as zinc pyrithione, a pyridinethione salt, a trihalocarbamide, triclosan, an azole compound, an antifungal polymer, allantoin, steroids such as topical corticosteroids, tar or polytar (coal tar), undecylenic acid, fumaric acid, an allylamine and mixtures thereof, ciclopirox, octopirox, piroctone olamine, clobetasol propionate, betamethasone valerate, tea tree oil, a mixed oil of thyme and catnip, topical antifungals such as selenium sulfide, imidazole (e.g. ketoconazole), hydroxypyridones (e.g. ciclopirox), naturopathic agents such as Melaleuca sp. oil, Aloe vera, and probiotic microorganisms. (Indian J. Dermatol, 2010 April-June; 55(2): 130-134).

In one aspect, the skin care benefit agent (active agent) consists of at least one microorganism of the Yarrowia genus, and/or fraction thereof, and/or cell lysate thereof, and/or fermentate thereof, and/or metabolite thereof. Skin care benefit agents include agents of at least one microorganism of the genus Yarrowia and/or a fraction thereof; and/or a cell lysate thereof, and/or fermentate thereof, and/or metabolite thereof providing growth inhibition of dandruff inducing microorganism via lipid consumption competition, lipase inhibitors, small molecules, or any one combination thereof. Skin care benefit agents of at least one microorganism of the genus Yarrowia and/or a fraction thereof, and/or a cell lysate thereof, and/or fermentate thereof; and/or metabolite thereof further include agents (actives) that removes biofilm of Malassezia species, and agents (actives) that prevent or reduce biofilm formation of Malassezia species.

In one aspect, the skin care benefit agent (active agent) consisting of at least one microorganism of the Yarrowia genus, and/or a fraction thereof, and/or a cell lysate thereof, and/or fermentate thereof; and/or metabolite thereof, is formulated in a skin care composition

It will be understood that the skin care composition for use in the present invention may comprise at least one microorganism of the genus Yarrowia, at least one metabolite of Yarrowia and/or at least one cell lysate of Yarrowia.

It will be further apparent that the skin care composition for use according to the present invention may comprise, for example, at least about 0.01%, about 0.05%, about 0.1%, about 0.2%, about 0.3%, about 0.4%, about 0.5%, about 0.6%, about 0.7%, about 0.8%, about 0.9%, about 1.0%, about 1.5%, about 2.0%, about 3.0%, about 4.0%, about 5.0%, about 6.0%, about 7.0%, about 8.0%, about 9.0%, about 10.0%, about 11.0%, about 12.0%, about 13.0%, about 14.0%, about 15.0%, about 16.0%, about 17.0%, about 18.0%, about 19.0%, about 20.0%, about 25.0%, about 30.0%, about 35.0%, about 40.0 about 45.0%, about 50.0% by weight of the Yarrowia microorganism(s), and/or a fraction thereof, and/or a cell lysate thereof, and/or fermentate thereof, and/or metabolite thereof.

In one embodiment, the composition is a skin care composition for use in the treatment of a scalp disorder, comprising an effective amount of at least one microorganism of the genus Yarrowia and/or a fraction thereof, and/or a cell lysate thereof, and/or fermentate thereof, and/or metabolite thereof, wherein said composition reduces and/or treats said scalp disorder.

In one embodiment, the composition is a skin care composition for use in the treatment of a scalp disorder, comprising an effective amount of at least one microorganism of the genus Yarrowia and/or a fraction thereof, and/or a cell lysate thereof, and/or fermentate thereof, and/or metabolite thereof, wherein said composition reduces and/or treats said scalp disorder.

In one aspect the scalp disorder is selected from the group consisting of a dandruff condition of the scalp (seborrheic dermatitis), unbalanced ecoflora of the scalp, discomfort of the scalp, tinea versicolor, dry skin, irritated skin, or any one combination thereof.

In one embodiment, the composition is a skin care composition for use in the treatment of a scalp disorder, comprising an effective amount of at least one microorganism of the genus Yarrowia and/or a fraction thereof, and/or a cell lysate thereof, and/or fermentate thereof, and/or metabolite thereof, wherein said composition reduces and/or treats said scalp disorder, wherein the composition degrades lipids selected from the group consisting of sapienic acid (C16:1 cis-6), palmitic acid (C16:0), myristic acid (C14:0), petroselinic acid (C18:1 cis-6), pentadecylic acid (C15:0), stearic acid (C18:0), lauric acid (C12:0), leis acid, and any one combination thereof.

In one embodiment, the composition is a skin care composition for use in the treatment of a scalp disorder, comprising an effective amount of at least one microorganism of the genus Yarrowia and/or a fraction thereof, and/or a cell lysate thereof, and/or fermentate thereof, and/or metabolite thereof, wherein said composition reduces and/or treats said scalp disorder, wherein the composition reduces the growth of Malassezia species.

In one embodiment, the skin care composition or skin care formulation is a lotion, a serum, a jelly, a cream, a gel, an emulsion, a mask, a patch, or a stick comprising one or more dermatologically or skin care acceptable components and at least about 1%, 2%, 3%, 4%, 5%, 6%, 7%, 8%, 9% up to 10% of the skin care formulation described herein on a weight basis relative to a total weight of said skin care formulation.

In some embodiments, the compositions of the invention may include isolated Yarrowia in combination with one or more dermatologically or skin care acceptable component carrier. The Yarrowia can be live or non-replicating, e.g., inactivated, for example, by heat-treatment. The dosage may vary, but can range from the equivalent of about 10² to about 10¹² cfu/g, e.g., 1×10² cfu/g, 5×10² cfu/g, 1×10³ cfu/g, 5×10³ cfu/g, 1×10⁴ cfu/g, 5×10⁴ cfu/g, 1×10⁵ cfu/g, 5×10⁵ cfu/g, 1×10⁶ cfu/g, 5×10⁶ cfu/g, 1×10⁷ cfu/g, 5×10⁷ cfu/g, 1×10⁸ cfu/g, 5×10⁸ cfu/g, 1×10⁹ cfu/g, 5×10⁹ cfu/g, 1×10¹⁰ cfu/g, 5×10¹⁰ cfu/g, 1×10¹¹ cfu/g, 5×10¹¹ cfu/g, 1×10¹² cfu/g of dry weight.

In some embodiments, the Yarrowia can be sterilized using conventional sterilization techniques before or after it is combined with the one or more dermatologically or skin care acceptable component

In one embodiment, the skin care composition is formulated in a skin care product/formulation for administration to the skin.

It will be further understood that the skin care composition for use in the present invention may further comprise one or more of probiotic bacteria in addition to the microorganism of the Yarrowia genus, and/or a fraction thereof, and/or a cell lysate thereof, and/or fermentate thereof, and/or metabolite thereof.

The skin care composition can comprise additional compounds selected from the group consisting of preservatives, pH adjusters, anti-oxidants and chelators.

Preservatives include but are not limited to parabens, sodium benzoate, potassium sorbate, phenyl ethyl alcohol, Lauryl ethyl arginate (LAE) and any combination thereof.

pH adjusters include but are not limited to weak acids, strong acids, any compound that can adjust the pH, such as but not limiting to citric acid, or any combination thereof.

The skin care compositions or any effective amount of said skin care composition described herein can be used in formulations and skin care products.

As used herein “skin care products” refer to products comprising an effective amount of the skin care compositions described herein, including but not limiting to cosmetic products, aqueous solutions, emulsions, serums, jellies, patches, lotions, topical moisturizers, creams, pastes, balms, ointments, pomades, gels, liquids, sprays, foam, kits, or any one combinations thereof.

In one embodiment, the skin care product is formulated for topical administration to the skin/scalp.

In one embodiment, the composition is a skin care product comprising an effective amount of a skin care composition for use in the treatment of a scalp disorder, comprising an effective amount of at least one microorganism of the genus Yarrowia and/or a fraction thereof, and/or a cell lysate thereof, and/or fermentate thereof, and/or metabolite thereof, wherein said composition reduces and/or treats said scalp disorder, and one or more dermatologically or skin care acceptable component.

In one embodiment, the skin care product is a lotion, a serum, a jelly, a cream, a gel, an emulsion, a mask, a patch, or a stick comprising one or more dermatologically or skin care acceptable components and at least about 1%, 2%, 3%, 4%, 5%, 6%, 7%, 8%, 9% up to 10% of the skin care formulation described herein on a weight basis relative to a total weight of said skin care formulation.

In one embodiment, the skin care product is a product comprising at least one microorganism of the genus Yarrowia, wherein the microorganism and/or the skin care composition comprising said microorganism is formulated in at least one form selected from the group consisting of a gel, an emulsion, a hydrogel, a loose or compact powder, a liquid suspension or solution, a spray solution, or any combination thereof.

The topical formulation for use in the present invention may be in any form suitable for application to the scalp or skin surface, such as a cream, lotion, sprays, solution, gel, ointment, paste, plaster, paint, bioadhesive, suspensions or the like, and/or may be prepared so as to contain liposomes, micelles, and/or microspheres. Such a formulation may be used in combination with an occlusive overlayer so that moisture evaporating from the body surface is maintained within the formulation upon application to the body surface and thereafter.

Topical formulations include those in which the active ingredient(s) is (are) dissolved or dispersed in a dermatological vehicle known in the art (e.g. aqueous or non-aqueous gels, ointments, water-in-oil or oil-in-water emulsions).

Constituents of such vehicles may comprise water; aqueous buffer solutions, non-aqueous solvents (such as ethanol, isopropanol, benzyl alcohol, 2-(2-ethoxyethoxy) ethanol, propylene glycol, propylene glycol monolaurate, glycofurol or glycerol), oils (e.g. a mineral oil such as a liquid paraffin, natural or synthetic triglycerides, or silicone oils such as dimethicone). Depending, inter glia, upon the nature of the formulation as well as its intended use and site of application, the dermatological vehicle employed may contain one or more components (for example, when the formulation is an aqueous gel, components in addition to water) selected from the following list: a solubilizing agent or solvent (e.g. a [symbol] -cyclodextrin, such as hydroxypropyl [symbol] -cyclodextrin, or an alcohol or polyol such as ethanol, propylene glycol or glycerol); a thickening agent (e.g. hydroxyethylcellulose, hydroxypropylcellulose, carboxymethylcellulose or carbomer); a gelling agent (e.g. a polyoxyethylene-polyoxypropylene copolymer); a preservative (e.g. benzyl alcohol, benzalkonium chloride, chlorhexidine, chlorbutol, a benzoate, potassium sorbate or EDTA or salt thereof); and pH buffering agent(s) (such as a mixture of dihydrogen phosphate and hydrogen phosphate salts, or a mixture of citric acid and a hydrogen phosphate salt).

A skin care product includes a liquid lotion (true solution) comprising water as a solvent and watersoluble additives (solutes), such as but not limiting to an active, a fragrance, a color, a preservative, a pH adjuster, a chelating agent, or any one combination thereof.

A skin care product includes a dispersion such as an emulsion (such as, but not limited to the following: liquid in liquid [water in oil W/O, O/W, W/O/W], suspension [solid/liquid or liquid/solid], aerosol [liquid/gas or solid/gas], foam/mousse [gas/liquid or gas/emulsion, or gas/solid]). An example of an Oil in Water [O/W] emulsion includes, but is not limited to a combination of a water phase; an emulsifier; a fatty phase and an at least one additive. The water phase can comprise water, humectants and stabilizing agents [such as, but not limiting to, synthetic polymers, carbomers, natural polymers, xanthan gum, acacia gum; carragheenan; gellan, or any one combination thereof). Emulsifiers include, but are not limited to, anionic emulsifiers, cationic emulsifiers, non-ionic emulsifiers, amphoteric emulsifiers, silicone emulsifiers), auto emulsifying agents. Fatty phases (lipophilic ingredients) include, but are not limited to, waxes, butter, fatty esters, triglycerides, vegetal oil, mineral oil (parffinum), silicones, and thickeners/oil jellifying agents. Additives include, but are not limited to, preservative, fragrance (most often lipophilic), color, anti-oxidant, chelating agent, actives, pH adjuster (citric acid, lactic acid, AHA), neutralizers/strong basic agent like NaOH, Trimethylamine (for acrylic polymers to jellify) and powders.

A skin care product includes an aqueous gel comprising a water phase (including water, humectants, actives), a jellifying agent (such as but not limited to synthetic polymers, natural polymers, xanthan gum, acacia gum, carragheenan, gellan) and an additive (such as but not limited to fragrance, high HLB surfactant, color, actives, preservative system, pH adjuster, neutralizing agent, powders).

A skin care product includes a cleansing/surfactant system (such as but not limited to a shampoo, shower gel, micellar water) comprising a water phase (water, humectants), a surfactant, an additive (such as but not limited to fragrance, high HLB surfactant, color, actives, preservative system, pH adjuster, neutralizing agent, powders) and optionally a jellifying agent (such as but not limited to synthetic polymers, natural polymers, xanthan gum, acacia gum, carragheenan, gellan).

As described herein, a skin care product or formulation comprising at least one microorganism of the Yarrowia genus, and/or a fraction thereof, and/or a cell lysate thereof, and/or fermentate thereof, and/or metabolite thereof as described herein, provides a skin care benefit selected from the group consisting of preventing a dandruff condition, reducing a dandruff condition, treatment of a dandruff condition, reducing the occurrence of Malassezia species (Malassezia spp.) on the skin/scalp, improving the barrier function of the skin, skin moisturizing (protecting the skin/scalp against dehydration by maintaining, restoring and/or strengthening the moisturization of the skin) or any one combination thereof.

A dermatologically or skin care acceptable carrier may also be incorporated in the skin care product (formulation) of the present invention and may be any carrier conventionally used in the art. Examples thereof include water, lower alcohols, higher alcohols, polyhydric alcohols, monosaccharides, disaccharides, polysaccharides, hydrocarbon oils, fats and oils, waxes, fatty acids, silicone oils, nonionic surfactants, ionic surfactants, silicone surfactants, and water-based mixtures and emulsion-based mixtures of such carriers.

The term “dermatologically acceptable” or “dermatologically acceptable carrier” or “skin care acceptable” or “skin care acceptable carrier” is used herein to refer to a compound or composition that may be incorporated into a dermatologically or skin care formulation without causing undesirable biological effects or unwanted interaction with other components of the formulation.

“Carriers” or “vehicles” as used herein refer to carrier materials suitable for incorporation in a topically applied composition. Carriers and vehicles useful herein include any such materials known in the art, which are nontoxic and do not interact with other components of the formulation in which it is contained in a deleterious manner.

The term “aqueous” refers to a formulation that contains water or that becomes water-containing following application to the skin or mucosal tissue.

Skin care products described herein may further comprise one or more dermatologically or skin care acceptable components known or otherwise effective for use skin care, provided that the optional components are physically and chemically compatible with the essential components described herein, or do not otherwise unduly impair product stability, aesthetics, or performance. Non-limiting examples of such optional components are disclosed in International Skin Care Ingredient Dictionary, Ninth Edition, 2002, and CTFA Skin Care Ingredient Handbook, Tenth Edition, 2004.

In one aspect, the dermatologically or skin care acceptable component is a dermatologically acceptable carrier comprising from about 10 wt. % to about 99.9 wt. %, alternatively from about 50 wt. % to about 95 wt. %, and alternatively from about 75 wt. % to about 95 wt. %, of a dermatologically acceptable carrier. Carriers suitable for use with the composition(s) may include, for example, those used in the formulation of mousses, tonics, gels, skin moisturizers and lotions. The carrier may comprise water; organic oils; silicones such as volatile silicones, amino or non-amino silicone gums or oils, and mixtures thereof; mineral oils; plant oils such as olive oil, castor oil, rapeseed oil, coconut oil, wheat germ oil, sweet almond oil; avocado oil, macadamia oil, apricot oil, safflower oil, candlenut oil, false flax oil, tamanu oil, lemon oil and mixtures thereof; waxes; and organic compounds such as C₂-C₁₀ alkanes, acetone, methyl ethyl ketone, volatile organic C₁-C₁₂ alcohols, esters of C₁-C₂₀ acids and of C₁-C₆ alcohols such as methyl acetate, butyl acetate, ethyl acetate, and isopropyl myristate, dimethoxyethane, diethoxyethane, C₁₀-C₃₀ fatty alcohols such as lauryl alcohol, cetyl alcohol, stearyl alcohol, and behenyl alcohol; C₁₀-C₃₀ fatty acids such as lauric acid and stearic acid; C₁₀-C₃₀ fatty amides such as lauric diethanolamide; C₁₀-C₃₀ fatty alkyl esters such as C₁₀-C₃₀ fatty alkyl benzoates; hydroxypropylcellulose, and mixtures thereof. In one aspect, the carrier comprises water, fatty alcohols, volatile organic alcohols, and mixtures thereof. Other carriers can be formulated by those of ordinary skill in the art.

The skin care products described herein may further comprise from about 0.1% to about 10%, and alternatively from about 0.2% to about 5.0%, of a gelling agent to help provide the desired viscosity to the composition(s). Non-limiting examples of suitable optional gelling agents include crosslinked carboxylic acid polymers; unneutralized crosslinked carboxylic acid polymers; unneutralized modified crosslinked carboxylic acid polymers; crosslinked ethylene/maleic anhydride copolymers; unneutralized crosslinked ethylene/maleic anhydride copolymers (e.g., EMA 81 commercially available from Monsanto); unneutralized crosslinked alkyl ether/′acrylate copolymers (e.g., SALCARE™ SC90 commercially available from Allied Colloids); unneutralized crosslinked copolymers of sodium polyacrylate, mineral oil, and PEG-1 trideceth-6 (e.g., SALCARE™ SC91 commercially available from Allied Colloids); unneutralized crosslinked copolymers of methyl vinyl ether and maleic anhydride (e.g., STABILEZE™ QM-PVM/MA copolymer commercially available from International Specialty Products); hydrophobically modified nonionic cellulose polymers; hydrophobically modified ethoxylate urethane polymers (e.g., UCARE™ Polyphobe Series of alkali swellable polymers commercially available from Union Carbide); and combinations thereof. In this context, the term “unneutralized” means that the optional polymer and copolymer gelling agent materials contain unneutralized acid monomers.

The dermatologically or skin care acceptable medium may contain a fatty substance in a proportion generally of from about 10 to about 90% by weight relative to the total weight of the product, where the fatty phase containing at least one liquid, solid or semi-solid fatty substance. The fatty substance includes, but is not limited to, oils, waxes, gums, and so-called pasty fatty substances. Alternatively, the products may be in the form of a stable dispersion such as a water-in-oil or oil-in-water emulsion. Additionally, the skin care products may contain one or more conventional skin care or dermatological additives or adjuvants, including but not limited to, antioxidants, preserving agents, fillers, surfactants, UVA and/or UVB sunscreens, fragrances, thickeners, wetting agents and anionic, nonionic or amphoteric polymers, and dyes or pigments (colorant agents).

The dermatologically acceptable carrier may be a moisturizer formulation containing at least one emulsifiers, at least one surfactant, or any combination thereof.

In one embodiment, the skin care product is a product comprising a first skin care composition and a second skin care composition, wherein the first skin care composition comprises an effective amount of a first active agent consisting of at least one microorganism of the genus Yarrowia and/or a fraction thereof, and/or a cell lysate thereof, and/or fermentate thereof, and/or metabolite thereof, wherein the second skin care composition comprises at least an effective amount of at least one second active agent (such as antidandruff active agents, skin conditioning agents, skin care active ingredient materials) for topical administration.

Skin care compositions and skin care products can further comprise skin care active ingredient materials including sun screen agents, moisturizers, humectants, benefiting agents skin, depositing agents such as surfactants, occlusive agents, moisture barriers, lubricants, emollients, anti-aging agents, antistatic agents, abrasive, antimicrobials, conditioners, exfoliants, fragrances, viscosifying agents, salts, lipids, phospholipids, vitamins, foam stabilizers, pH modifiers, preservatives, suspending agents; silicone oils, silicone derivatives, essential oils, oils, fats, fatty adds, fatty add esters, fatty alcohols, waxes, polyols, hydrocarbons, and mixtures thereof.

Other ingredients that may be included in a skin care composition or skin care product include, without limitation, at least one active ingredient for the treatment or prevention of skin ailments, providing a skin care effect, or for providing a moisturizing benefit to skin, such as zinc oxide, petrolatum, white petrolatum, mineral oil, cod liver oil, lanolin, dimethicone, hard fat, vitamin A, allantoin, calamine, kaolin, glycerin, or colloidal oatmeal, and combinations of these, one or more natural moisturizing factors (such as ceramides, hyaluronic acid, glycerin, squalane, amino acids, cholesterol, fatty acids, triglycerides, phospholipids, glycosphingolipids, urea, linoleic acid, glycosaminoglycans, mucopolysaccharide, sodium lactate; or sodium pyrrolidone carboxylate, for example), glycerides, apricot kernel oil, canola oil, squalane, squalene, coconut oil, corn oil, jojoba oil, jojoba wax, lecithin, olive oil, safflower oil, sesame oil, shea butter, soybean oil, sweet almond oil, sunflower oil, tea tree oil, shea butter, palm oil, cholesterol, cholesterol esters, wax esters, fatty acids, and orange oil.

Any number of dermatologically acceptable materials commonly used in skin care products may also be incorporated into the present skin care products such as skin conditioning agents and skin colorants.

Skin conditioning agents as herein defined include, but are not limited to astringents, which tighten skin; exfoliants, which remove dead skin cells; emollients; which help maintain a smooth, soft, pliable appearance; humectants, which increase the water content of the top layer of skin; occlusives, which retard evaporation of water from the skin's surface; and miscellaneous compounds that enhance the appearance of dry or damaged skin or reduce flaking and restore suppleness. Skin conditioning agents are well known in the art, see for example Green et al. (WO01/07009), and are available commercially from various sources. Suitable examples of skin conditioning agents include, but are not limited to, lactobionic acid, gluconic acid, alpha-hydroxy acids, beta-hydroxy acids, polyols, hyaluronic acid, D,L-panthenol, polysalicylates, vitamin A palmitate, vitamin E acetate, glycerin, sorbitol, silicones, silicone derivatives, lanolin, natural oils, xylitol, fucose, rhamnose, and triglyceride esters. The skin conditioning agents may include polysalicylates, propylene glycol (CAS No. 57-55-6; Dow Chemical, Midland, Mich.), glycerin (CAS No. 56-81-5, Proctor & Gamble Co., Cincinnati, Ohio), glycolic acid (CAS No. 79-14-1, DuPont Co., Wilmington, Del.); lactic acid (CAS No. 50-21-5, Alfa Aesar, Ward Hill, Mass.), malic acid (CAS No. 617-48-1, Alfa Aesar), citric add (CAS No. 77-92-9, Alfa Aesar), tartaric acid (CAS NO. 133-37-9; Alfa Aesar), glucaric acid (CAS No. 87-73-0), galactaric acid (CAS No. 526-99-8), 3-hydroxyvaleric acid (CAS No. 10237-77-1); salicylic acid (CAS No. 69-72-7, Alfa Aesar), and 1,3 propanediol (CAS No. 504-63-2, DuPont Co., Wilmington, Del.). Polysalicylates may be prepared by the method described by White et al. in U.S. Pat. No. 4,855,483, incorporated herein by reference. Glucaric acid may be synthesized using the method described by Merbouh et al. (Carbohydr. Res, 336:75-78 (2001). The 3-hydroxyvaleric acid may be prepared as described by Bramucci in published international patent application number WO 02/012530.

Skin care compositions and skin care products can comprise skin care additives such as, but not limiting to, colorants/dyes, fragrances; actives; preservatives, pH adjusters, chelators, and anti-oxidants.

In one embodiment, the skin care product is a product comprising a first skin care composition and a second skin care composition, wherein the first skin care composition comprises an effective amount of a first active agent consisting of at least one microorganism of the genus Yarrowia and/or a fraction thereof, and/or a cell lysate thereof, and/or fermentate thereof, and/or metabolite thereof; wherein the second skin care composition comprises at least an effective amount of at least one second active agent selected from antidandruff active agents, for topical administration. Examples of such antidandruff active agents include keratolytic agents such as salicylic acid and sulphur in its various forms; regulators of keratinization such as zinc pyrithione, a pyridinethione salt, a trihalocarbamide, triclosan, an azole compound, an antifungal polymer; allantoin, steroids such as topical corticosteroids; tar or polytar (coal tar), undecylenic acid, fumaric acid, an allylamine and mixtures thereof, ciclopirox, octopirox, piroctone olamine, clobetasol propionate, betamethasone valerate, tea tree oil, a mixed oil of thyme and catnip, topical antifungals such as selenium sulfide, imidazole (e.g. ketoconazole), hydroxypyridones (e.g. ciclopirox), naturopathic agents such as Melaleuca sp. oil, Aloe vera, and probiotic microorganisms. (Indian J. Dermatol, 2010 April-June; 55(2): 130-134).

In one embodiment, the skin care product is a product comprising a first skin care composition and a second skin care composition, wherein the first skin care composition comprises an effective amount of a first active agent consisting of at least one microorganism of the genus Yarrowia and/or a fraction thereof, and/or a cell lysate thereof, and/or fermentate thereof, and/or metabolite thereof; wherein the second skin care composition comprises at least an effective amount of at least one second active agent selected from antidandruff active agents, for topical administration, wherein the first skin care composition is formulated in at least one form selected from the group consisting of a gel, an emulsion, a hydrogel, a loose or compact powder, a liquid suspension or solution, or a spray solution.

Skin care compositions and skin care products described herein can also be part of a kit for providing one or more skin care benefits such as, but not limiting to, a kit for preventing or reducing a dandruff condition

In one aspect the kit is a kit comprising the at least one microorganism of the genus Yarrowia for the treatment of a dandruff condition and written instructions for administration to the subject in need.

In one aspect the kit is a kit comprising a skin care product for the treatment of a dandruff condition of a subject in need, wherein said skin care product comprises at least one microorganism of the genus Yarrowia, and written instructions for administration said skin care product to the subject in need.

### Methods for Microbial Treatment of a Scalp Disorder

Further provided herein are methods for treating a scalp disorder in a subject in need thereof, comprising administering an effective amount of at least one microorganism of the genus Yarrowia and/or a fraction thereof, and/or a cell lysate thereof, and/or fermentate thereof, and/or metabolite thereof, to said subject.

In one aspect, the scalp disorder is selected from the group consisting of a dandruff condition of the scalp (seborrheic dermatitis), unbalanced ecoflora of the scalp, discomfort of the scalp, tinea versicolor, dry skin, irritated skin, or any one combination thereof.

In one aspect, the microorganism is administered topically.

In one aspect, the microorganism is a microorganism of the genus Yarrowia selected from the group consisting of Yarrowia lipolytica ATCC 20362, Yarrowia lipolytica ATCC 9773, Yarrowia lipolytica ATCC 18942, Yarrowia lipolytica ATCC 20177, Yarrowia lipolytica CBS2073, Yarrowia lipolytica Phaff #50-47, or any one combination thereof.

In one embodiment, the method is a method for treating a scalp disorder in a subject in need thereof, comprising administering an effective amount of at least one microorganism of the genus Yarrowia and/or a fraction thereof, and/or a cell lysate thereof, and/or fermentate thereof, and/or metabolite thereof, to said subject.

In one embodiment, the method is a method for treating a scalp disorder in a subject in need thereof, comprising administering a Yarrowia lipolytica and/or, a fraction thereof, and/or a cell lysate thereof, and/or fermentate thereof, and/or metabolite thereof to said subject.

The skin care compositions and skin care products described herein can be used in methods for treating a scalp disorder.

In one embodiment, the method is a method for treating a scalp disorder in a subject in need thereof, comprising administering a skin care composition for use in the treatment of a scalp disorder, wherein said skin care composition comprises an effective amount of at least one microorganism of the genus Yarrowia and/or a fraction thereof, and/or a cell lysate thereof, and/or fermentate thereof, and/or metabolite thereof, wherein said composition reduces and/or treats said scalp disorder.

In one embodiment, the method is a method for treating a scalp disorder in a subject in need thereof, comprising administering a skin care composition for use in the treatment of a scalp disorder, wherein said skin care composition comprises an effective amount of Yarrowia lipolytica and/or a fraction thereof, and/or a cell lysate thereof, and/or fermentate thereof, and/or metabolite thereof, wherein said composition reduces and/or treats said scalp disorder.

In one embodiment, the method is a method for treating a scalp disorder in a subject in need thereof, comprising administering a skin care product for use in the treatment of a scalp disorder, wherein said skin care product comprises an effective amount of at least one microorganism of the genus Yarrowia and/or a fraction thereof, and/or a cell lysate thereof, and/or fermentate thereof, and/or metabolite thereof; wherein said skin care product reduces and/or treats said scalp disorder.

In one embodiment, the method is a method for treating a scalp disorder in a subject in need thereof, comprising administering a skin care product for use in the treatment of a scalp disorder, wherein said skin care product comprises an effective amount of s at least one Yarrowia lipolytica and/or a fraction thereof, and/or a cell lysate thereof, and/or fermentate thereof, and/or metabolite thereof, wherein said skin care product reduces and/or treats said scalp disorder.

In one aspect, the skin care composition or skin care product is administered topically.

Further provided herein is a kit, comprising the compositions described herein and instructions for the use thereof to treat a skin condition. In some embodiments, the kit further comprises one or more applicator configured to apply the composition.

Methods for Treating and/or Reducing a Dandruff Condition.

Further provided herein are methods for treating a dandruff condition in a subject in need thereof, comprising administering an effective amount of at least one microorganism of the genus Yarrowia and/or a fraction thereof, and/or a cell lysate thereof, and/or fermentate thereof, and/or metabolite thereof, to said subject.

In one embodiment, the method is a method for treating and/or reducing a dandruff condition of the scalp in a subject in need thereof, comprising administering an effective amount of at least one microorganism of the genus Yarrowia and/or a fraction thereof, and/or a cell lysate thereof, and/or fermentate thereof, and/or metabolite thereof, to said subject.

In one embodiment, the method is a method for treating and/or reducing a dandruff condition of the scalp in a subject in need thereof, comprising administering an effective amount of at least one Yarrowia lipolytica and/or a fraction thereof, and/or a cell lysate thereof, and/or fermentate thereof, and/or metabolite thereof, to said subject.

In one aspect, the microorganism is administered topically.

In one aspect, the microorganism and/or a fraction thereof, and/or a cell lysate thereof, and/or fermentate thereof, and/or metabolite thereof is a microorganism of the genus Yarrowia is selected from the group consisting of Yarrowia lipolytica ATCC 20362, Yarrowia lipolytica ATCC 9773, Yarrowia lipolytica ATCC 18942, Yarrowia lipolytica ATCC 20177, Yarrowia lipolytica CB32073, Yarrowia lipolytica Phaff #50-47, or any one combination thereof.

In one embodiment, the method is a method for treating and/or reducing a dandruff condition of the scalp in a subject in need thereof, comprising administering an effective amount of at least one microorganism of the genus Yarrowia and/or a fraction thereof, and/or a cell lysate thereof, and/or fermentate thereof, and/or metabolite thereof, to said subject, wherein the at least one microorganism of Yarrowia and/or a fraction thereof, and/or a cell lysate thereof, and/or fermentate thereof, and/or metabolite thereof, is formulated in a single composition.

In one aspect, the microorganism and/or a fraction thereof, and/or a cell lysate thereof, and/or fermentate thereof, and/or metabolite thereof is administered topically.

In one embodiment, the method is a method for treating and/or reducing a dandruff condition of the scalp in a subject in need thereof, comprising administering an effective amount of at least one microorganism of the genus Yarrowia and/or a fraction thereof, and/or a cell lysate thereof, and/or fermentate thereof, and/or metabolite thereof, to said subject, wherein the at least one microorganism of the genus Yarrowia and/or a fraction thereof, and/or a cell lysate thereof, and/or fermentate thereof, and/or metabolite thereof, is formulated in a single composition, and wherein the composition is administered to the subjects' skin or scalp.

In one embodiment, the method is a method for treating and/or reducing a dandruff condition of the scalp in a subject in need thereof, comprising administering an effective amount of at least one microorganism of the genus Yarrowia and/or a fraction thereof, and/or a cell lysate thereof, and/or fermentate thereof, and/or metabolite thereof, to said subject, wherein the at least one microorganism of the genus Yarrowia and/or a fraction thereof, and/or a cell lysate thereof, and/or fermentate thereof, and/or metabolite thereof, is formulated in a single composition, wherein the composition further comprises a compound selected from the group consisting of a skin care excipient, butyric acid, glucose, glycogen, magnesium ascorbyl phosphate, cetyl alcohol, dimethicone, isopropyl myristate, glycerol, propylene glycol, Quaternium-52, ethanol or any one combination thereof.

The skin care compositions and skin care products described herein can be used in methods for treating a dandruff condition.

In one embodiment, the method is a method for treating and/or reducing a dandruff condition of the scalp in a subject in need thereof, comprising administering a skin care composition for use in the treatment of a dandruff condition, wherein said skin care composition comprises an effective amount of at least one microorganism of the genus Yarrowia and/or a fraction thereof, and/or a cell lysate thereof, and/or fermentate thereof, and/or metabolite thereof, wherein said composition reduces and/or treats said dandruff condition.

In one embodiment, the method is a method for treating and/or reducing a dandruff condition of the scalp in a subject in need thereof, comprising administering a skin care product for use in the treatment of a dandruff condition, wherein said skin care composition comprises an effective amount of at least one microorganism of the genus Yarrowia sand/or a fraction thereof, and/or a cell lysate thereof, and/or fermentate thereof, and/or metabolite thereof, wherein said composition reduces and/or treats said dandruff condition.

In one embodiment, the method is a method for treating and/or reducing a dandruff condition of the scalp in a subject in need thereof, comprising administering an effective amount of at least one microorganism of the genus Yarrowia and/or a fraction thereof, and/or a cell lysate thereof, and/or fermentate thereof, and/or metabolite thereof, to said subject, wherein the at least one microorganism of the genus Yarrowia and/or a fraction thereof, and/or a cell lysate thereof, and/or fermentate thereof, and/or metabolite thereof, reduces the growth of Malassezia species.

In one embodiment, the method is a method for treating and/or reducing a dandruff condition of the scalp in a subject in need thereof, comprising administering an effective amount of at least one microorganism of the genus Yarrowia and/or a fraction thereof, and/or a cell lysate thereof, and/or fermentate thereof, and/or metabolite thereof, to said subject, wherein the at least one microorganism of the genus Yarrowia and/or a fraction thereof, and/or a cell lysate thereof, and/or fermentate thereof, and/or metabolite thereof removes biofilm of Malassezia species.

In one embodiment, the method is a method for treating and/or reducing a dandruff condition of the scalp in a subject in need thereof, comprising administering an effective amount of at least one microorganism of the genus Yarrowia and/or a fraction thereof, and/or a cell lysate thereof, and/or fermentate thereof, and/or metabolite thereof, to said subject, wherein the at least one microorganism of the genus Yarrowia and/or a fraction thereof, and/or a cell lysate thereof, and/or fermentate thereof, and/or metabolite thereof prevents or reduces biofilm formation of Malassezia species.

In one aspect, the method is a method for treating and/or reducing a dandruff condition of the scalp in a subject in need thereof, comprising topically administering a skin care product comprising a skin care composition described herein.

In one aspect, the method is a method for treating and/or reducing a dandruff condition of the scalp in a subject in need thereof, comprising topically administering a skin care product comprising a skin care composition described herein, wherein the skin care product is a selected from the group consisting of a lotion, a serum, a jelly, a cream, a gel, an emulsion, a solid cosmetic, a mask, a patch, and a stick comprising at least 1%, 2%, 3%, 4% up to 5% of said skin care composition on a weight basis relative to a total weight of said skin care product.

In one embodiment, the method is a method for treating and/or reducing a dandruff condition of the scalp in a subject in need thereof, comprising administering an effective amount of at least one microorganism of the genus Yarrowia and/or a fraction thereof, and/or a cell lysate thereof, and/or fermentate thereof, and/or metabolite thereof, to said subject.

In one aspect, the methods described herein for treating a dandruff condition inhibit Malassezia species

In one aspect, the methods described are methods wherein the at least one microorganism of the genus Yarrowia degrades lipids selected from the group consisting of sapienic acid (C16:1 ds-6), palmitic add (C16:0), myristic acid (C14:0), petroselinic acid (C18:1 cis-6), pentadecylic acid (C15:0), stearic acid (C18:0), auric acid (C12:0), oleic acid, and any one combination thereof.

### General Definitions

The disclosures of all cited patent and non-patent literature are incorporated herein by reference in their entirety.

In this disclosure, a number of terms and abbreviations are used. The following definitions apply unless specifically stated otherwise.

As used herein, the articles “a”, “an”, and “the” preceding an element or component of the invention are intended to be nonrestrictive regarding the number of instances (i.e., occurrences) of the element or component. Therefore “a”, “an”, and “the” should be read to include one or at least one, and the singular word form of the element or component also includes the plural unless the number is obviously meant to be singular.

When an amount, concentration, or other value or parameter is given either as a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope be limited to the specific values recited when defining a range.

The use of numerical values in the various ranges specified in this application, unless expressly indicated otherwise, are stated as approximations as though the minimum and maximum values within the stated ranges were both proceeded by the word “about”. In this manner, slight variations above and below the stated ranges can be used to achieve substantially the same results as values within the ranges. Also, the disclosure of these ranges is intended as a continuous range including each and every value between the minimum and maximum values. As used herein, the term “about” modifying the quantity of an ingredient or reactant employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities.

As used herein “administer” or “administering” is meant the action of introducing one or more microorganism (microbial strain), skin care composition(s), skin care formulation(s) and/or skin care product(s) to a subject in need for treatment of a scalp disorder.

Administering one or more microorganism (microbial strain), skin care composition(s), skin care formulation(s) and/or skin care product(s) to a subject includes applying or introducing one or more microorganism (microbial strain), skin care composition(s), skin care formulation(s) and/or skin care product(s) to a scalp, a skin surface, and to in-vitro or in-vivo skin cells.

As used herein, the term “biological contaminants” refers to one or more unwanted and/or pathogenic biological entities including, but not limited to, microorganisms, spores, viruses, prions, and mixtures thereof.

As used herein, the term “comprising” means the presence of the stated features, integers, steps, or components as referred to in the claims, but that it does not preclude the presence or addition of one or more other features, integers, steps, components or groups thereof. The term “comprising” is intended to include embodiments encompassed by the terms “consisting essentially of” and “consisting of”. Similarly, the term “consisting essentially of” is intended to include embodiments encompassed by the term “consisting of”.

As used herein, the term “embodiment” or “disclosure” is not meant to be limiting, but applies generally to any of the embodiments defined in the claims or described herein. These terms are used interchangeably herein.

As used herein, the term “excipient” refers to inactive substance used as a carrier for active ingredients, in a formulation. The excipient may be used to stabilize the active ingredient in a formulation, such as the storage stability of the active ingredient. Excipients are also sometimes used to bulk up formulations that contain active ingredients. An “active ingredient” includes a skin care benefit agent as described herein.

As used herein, the term “effective amount” refers to the amount sufficient to obtain the desired effect. A desired effect includes the prevention, reduction and/or treatment of a scalp disorder, such as the prevention, reduction and or treatment of dandruff condition.

As used herein, “prevent,” “preventing,” “prevention” and grammatical variations thereof refers to a method of partially or completely delaying or precluding the onset or recurrence of a disorder or condition (such as a scalp disorder) and/or one or more of its attendant symptoms or barring a subject from acquiring or reacquiring a disorder or condition or reducing a subject's risk of acquiring or reacquiring a disorder or condition or one or more of its attendant symptoms.

As used herein, the term “reducing”, “reduces” and grammatical variations thereof in relation to a particular trait, characteristic, feature, biological process, or phenomena refers to a decrease in the particular trait, characteristic, feature, biological process, or phenomena. The trait, characteristic, feature, biological process, or phenomena can be decreased by 5%, 10%, 15%, 20%, 25%, 30%, 35%, 40%, 45%, 50%, 55%, 60%, 65%, 70%, 75%, 80%, 85%, 90%, 95%, 100% or greater than 100%.

The terms “percent by weight”, “weight percentage (wt. %)” and “weight-weight percentage (% w/w)” are used interchangeably herein. Percent by weight refers to the percentage of a material on a mass basis as it is comprised in a composition, mixture, solution or product.

The term “163 rRNA” or “163 ribosomal RNA” means the rRNA constituting the small subunit of prokaryotic ribosomes. In bacteria, this sequence can be used to identify and characterize operational taxonomic units.

The term “ITS” or “Internal Transcribed Spacers” are regions within the ribosomal transcript that are excised and degraded during maturation. Their sequences can be used for phylogenetic analysis and/or identification of fungi or yeast.

The terms moisturizer, a lotion or a body lotion refer to a low to medium-viscosity emulsion of oil and water, most often oil-in-water but possibly water-in-oil with the primary benefit in a skin care application to hydrate the skin or to reduce its water loss. Nearly all moisturizers contain a combination of emollients, occlusives, and humectants. Emollients, which are mainly lipids and oils, hydrate and improve the appearance of the skin. A wide variety of suitable emollients is known and maybe used herein (International Skin Care Ingredient Dictionary and Handbook, eds. Wenninger and McEwen, pp, 1656-61, 1626, and 1654-55 (The Skin care, Toiletry, and Fragrance Assoc., Washington, D.C., 7th Edition, 1997) (referred to as “ICI Handbook”) contains numerous examples of suitable materials). Occlusives such as petrolatum, lanolin and bee wax reduce transepidermal water loss by creating hydrophobic barrier over the skin. Humectants such as glycerol and urea able to attract water from the external environment and enhance water absorption from the dermis into the epidermis. In addition, the moisturizer formulations may contain emulsifiers to maintain stability of emulsions, and use thickeners to achieve desired viscosity and skin feel. A wide variety of other ingredients such as fragrances, dyes, preservatives, therapeutic agents, proteins and stabilizing agents are commonly added for other consumer preferred attributes.

The term “percent (%) sequence identity” or “percent (%) sequence similarity,” as used herein with respect to a reference sequence is defined as the percentage of nucleotide residues in a candidate sequence that are identical to the residues in the reference polynucleotide sequence after optimal alignment of the sequences and introducing gaps, if necessary, to achieve the maximum percent sequence identity.

A microbial “strain” as used herein refers to a microorganism (such as a bacterium or fungus) which remains genetically unchanged when grown or multiplied. The multiplicity of identical microbes is included.

As used herein, the term a “biologically pure strain” means a strain containing no other microbial strains in quantities sufficient to interfere with replication of the strain or to be detectable by normal techniques. “Isolated” when used in connection with the organisms and cultures described herein includes not only a biologically pure strain, but also any culture of organisms which is grown or maintained other than as it is found in nature.

In one aspect the skin cells described herein are mammalian skin cells, such as human or animal skin cells.

The term “sequence identity” or “sequence similarity” as used herein, means that two polynucleotide sequences, a candidate sequence and a reference sequence, are identical (i.e. 100% sequence identity) or similar (i.e. on a nucleotide-by-nucleotide basis) over the length of the candidate sequence. In comparing a candidate sequence to a reference sequence, the candidate sequence may comprise additions or deletions (i.e. gaps) as compared to the reference sequence (which does not comprise additions or deletions) for optimal alignment of the two sequences. Optimal alignment of sequences for determining sequence identity may be conducted using the any number of publicly available local alignment algorithms known in the art such as ALIGN or Megalign (DNASTAR), or by inspection.

It is intended that every maximum numerical limitation given throughout this specification includes every lower numerical limitation, as if such lower numerical limitations were expressly written herein, Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Unless defined otherwise herein, all technical and scientific terms used herein have the same meaning as commonly understood by one of ordinary skill in the art to which this invention pertains.

Non-limiting examples of compositions and methods disclosed herein include:

1. A skin care composition for use in the treatment of a scalp disorder, comprising an effective amount of at least one microorganism of the genus Yarrowia and/or a fraction thereof, and/or a cell lysate thereof, and/or fermentate thereof, and/or metabolite thereof, wherein said composition reduces and/or treats said scalp disorder.
2. The skin care composition of embodiment 1, wherein the scalp disorder is selected from the group consisting of a dandruff condition of the scalp (seborrheic dermatitis), unbalanced ecoflora of the scalp, discomfort of the scalp, tinea versicolor, dry skin, irritated skin, or any one combination thereof.

3. The skin care composition of embodiment 1, further comprising one or more antidandruff active agents.

4. The skin care composition of embodiment 1, wherein the composition degrades lipids selected from the group consisting of sapienic acid (C16:1 cis-6), palmitic acid (C16:0), myristic acid (C14:0), petroselinic acid (C18:1 cis-6), pentadecylic acid (C15:0), stearic acid (C18:0), lauric acid (C12:0), oleic acid, and any one combination thereof.
5. The skin care composition of embodiment 1, wherein the composition reduces the growth of Malassezia species.
6. The skin care composition of claim 1, wherein the composition removes biofilm of Malassezia species.
7. The skin care composition of claim 1, wherein the composition prevents or reduces biofilm formation of Malassezia species
8. The skin care composition of embodiment 1, further comprising at least one additional compound selected from the group consisting of an excipient, a preservative, a pH adjuster.
9. The skin care composition of embodiment 1, comprising at least one microorganism of the genus Yarrowia selected from the group consisting of Yarrowia lipolytica ATCC 20362, Yarrowia lipolytica ATCC 9773, Yarrowia lipolytica ATCC 18942, Yarrowia lipolytica ATCC 20177, Yarrowia lipolytica CBS2073, Yarrowia lipolytica Phaff #50-47, or any one combination thereof.
10. Use of an effective amount of the skin care composition of any preceding embodiment in a skin care product.
11. A skin care product comprising an effective amount of the skin care composition of any one of embodiment 1-9 and one or more dermatologically or skin care acceptable component.
11b. The skin care product of embodiment 11, wherein the product is formulated for topical administration.
12. The skin care product of embodiment 11, wherein said effective amount of the skin care composition is at least about 1%, 2%, 3%, 4%, 5%, 6%, 7%, 8%, 9% up to 10% on a weight basis relative to a total weight of said skin care product.
12b. A skin care product comprising a first skin care composition and a second skin care composition, wherein the first skin care composition comprises an effective amount of a first active agent consisting of at least one microorganism of the genus Yarrowia and/or a fraction thereof, and/or a cell lysate thereof, and/or fermentate thereof, and/or metabolite thereof; wherein the second skin care composition comprises at least an effective amount of at least one second active agent selected from antidandruff active agents for topical administration.
12c. The skin care product of embodiment 10b, wherein the first skin care composition is formulated in at least one form selected from the group consisting of, a gel, an emulsion, a hydrogel, a loose or compact powder, a liquid suspension or solution, or a spray solution.
12d. The skin care product of embodiment 10b, wherein the at least one second composition comprises at least one member selected from the group consisting of a hair lotion, a shampoo, a hair conditioner, a detangler, a hair cream or gel, a styling lacquer, a hairsetting lotion, a treating lotion, a dye composition, a hair-restructuring lotion, a permanent-waving composition, a lotion or gel for combating hair loss, an antiparasitic shampoo or a medicated shampoo, and a scalp care product.
13. A method for treating a scalp disorder in a subject in need thereof, comprising administering an effective amount of at least one microorganism of the genus Yarrowia and/or a fraction thereof, and/or a cell lysate thereof, and/or fermentate thereof, and/or metabolite thereof to said subject.
13b. A method for treating a scalp disorder in a subject in need thereof, comprising administering an effective amount of at least one Yarrowia lipolytica and/or, a fraction thereof, and/or a cell lysate thereof, and/or fermentate thereof, and/or metabolite thereof to said subject. 13c. A method for treating a scalp disorder in a subject in need thereof, comprising topically administering a skin care product comprising an effective amount of at least one microorganism of the genus Yarrowia and/or a fraction thereof, and/or a cell lysate thereof, and/or fermentate thereof, and/or metabolite thereof, to said subject.
14. The method according to embodiment 13, wherein the scalp disorder is selected from the group consisting of a dandruff condition of the scalp (seborrheic dermatitis), unbalanced ecoflora of the scalp, discomfort of the scalp, tinea versicolor, dry skin, irritated skin, or any one combination thereof.
15. The method according to embodiment 13, wherein the microorganism and/or a fraction thereof, and/or a cell lysate thereof, and/or fermentate thereof, and/or metabolite is administered topically.
15B. The method according to embodiment 13c, wherein the microorganism and/or a fraction thereof, and/or a cell lysate thereof, and/or fermentate thereof, and/or metabolite thereof is administered topically.
16. A method for treating and/or reducing a dandruff condition of the scalp in a subject in need thereof, comprising administering an effective amount of at least one microorganism of the genus Yarrowia and/or a fraction thereof, and/or a cell lysate thereof, and/or fermentate thereof, and/or metabolite thereof to said subject.
16. A method for treating and/or reducing a dandruff condition of the scalp in a subject in need thereof, comprising topically administering a skin care product comprising an effective amount of at least one microorganism of the genus Yarrowia and/or a fraction thereof, and/or a cell lysate thereof, and/or fermentate thereof, and/or metabolite thereof, to said subject.
17. The method according to embodiment 16, wherein the microorganism and/or, the fraction thereof, and/or the cell lysate thereof, and/or metabolite thereof is administered topically.
18. The method of embodiment 16, wherein the at least one microorganism of the genus Yarrowia and/or a fraction thereof, and/or a cell lysate thereof, and/or fermentate thereof, and/or metabolite thereof, is formulated in a single composition.
18b. The method of embodiment 16, wherein the at least one microorganism of the genus Yarrowia and/or a fraction thereof, and/or a cell lysate thereof, and/or fermentate thereof, and/or metabolite thereof, and/or metabolite thereof is in a composition further comprising at least one or more dermatologically or skin care acceptable component.
19. The method of embodiment 16, wherein the composition is administered to the subject's skin or scalp.
20. The method of embodiment 16, wherein the least one microorganism of the genus Yarrowia is selected from the group consisting of Yarrowia lipolytica ATCC 20362, Yarrowia lipolytica ATCC 9773, Yarrowia lipolytica ATCC 18942, Yarrowia lipolytica ATCC 20177, Yarrowia lipolytica CBS2073, Yarrowia lipolytica Phaff #50-47, or any one combination thereof.
21. The method of embodiment 16, wherein the at least one microorganism of the genus Yarrowia and/or a fraction thereof, and/or a cell lysate thereof, and/or fermentate thereof, and/or metabolite thereof inhibits Malassezia species.
21b. The method of embodiment 16, wherein the at least one microorganism of the genus Yarrowia and/or a fraction thereof, and/or a cell lysate thereof, and/or fermentate thereof, and/or metabolite thereof, reduces the growth of Malassezia species.
21c. The method of embodiment 16, wherein a fraction of the at least one microorganism of the genus Yarrowia reduces the growth of Malassezia species.
21d. The method of embodiment 16, wherein a cell lysate of the at least one microorganism of the genus Yarrowia reduces the growth of Malassezia species.
21e. The method of embodiment 16, wherein a fermentate of the at least one microorganism of the genus Yarrowia reduces the growth of Malassezia species.

21f. The method of embodiment 16, wherein a metabolite of the at least one microorganism of the genus Yarrowia reduces the growth of Malassezia species.

22. The method of embodiment 16, wherein the at least one microorganism of the genus Yarrowia and/or a fraction thereof, and/or a cell lysate thereof, and/or fermentate thereof, and/or metabolite thereof removes biofilm of Malassezia species.

22b. The method of embodiment 16, wherein a fraction of the at least one microorganism of the genus Yarrowia removes biofilm of Malassezia species.
22c. The method of embodiment 16, wherein a cell lysate of the at least one microorganism of the genus Yarrowia removes biofilm of Malassezia species.
22d. The method of embodiment 16, wherein a fermentate of the at least one microorganism of the genus Yarrowia removes biofilm of Malassezia species.
22e. The method of embodiment 16, wherein a metabolite of the at least one microorganism of the genus Yarrowia removes biofilm of Malassezia species.
23. The method of embodiment 16, wherein the at least one microorganism of the genus Yarrowia and/or a fraction thereof, and/or a cell lysate thereof, and/or fermentate thereof, and/or metabolite thereof prevents or reduces biofilm formation of Malassezia species.
23b. The method of embodiment 16, wherein a fraction of the at least one microorganism of the genus Yarrowia prevents or reduces biofilm formation of Malassezia species.
23c. The method of embodiment 16, wherein a cell lysate of the at least one microorganism of the genus Yarrowia prevents or reduces biofilm formation of Malassezia species.
23d. The method of embodiment 16, wherein a fermentate of the at least one microorganism of the genus Yarrowia prevents or reduces biofilm formation of Malassezia species.
23e. The method of embodiment 16, wherein a metabolite of the at least one microorganism of the genus Yarrowia prevents or reduces biofilm formation of Malassezia species.
24. The method of embodiment 16, wherein the at least one microorganism of the genus Yarrowia and/or a fraction thereof, and/or a cell lysate thereof, and/or fermentate thereof, and/or metabolite thereof degrades lipids selected from the group consisting of Sapienic acid (C16:1 cis-6), Palmitic add (C16:0), Myristic add (C14:0), Petroselinic add (C18:1 cis-6), Pentadecylic add (C15:0), Stearic add (C18:0), Lauric add (C12:0), Oleic add, and any one combination thereof.
25. The method of any one of embodiments 16-24, wherein the composition is administered as a skin care product, wherein said skin care product is a lotion, a serum, a jelly, a cream, a gel, an emulsion, a mask, a patch, or a stick comprising at least 1%, 2%, 3%, 4% up to 5% of said skin care composition on a weight basis relative to a total weight of said skin care product.
26. A method for treating and/or reducing a dandruff condition of the scalp in a subject in need thereof, comprising topically administering a skin care product comprising a skin care composition of any one of embodiments 1-9 to said subject.
27. The method of claim 26, wherein the skin care product is a selected from the group consisting of a lotion, a serum, a jelly, a cream, a gel, an emulsion, a solid cosmetic, a mask, a patch, and a stick comprising at least 1%, 2%, 3%, 4% up to 5% of said skin care composition on a weight basis relative to a total weight of said skin care product.
28. A method for treating and/or reducing a dandruff condition of the scalp in a subject in need thereof, comprising administering an effective amount of a first cosmetic active agent and an effective amount of at least one second cosmetic active agent, wherein the first cosmetic active agent consist of at least one microorganism of the genus Yarrowia and/or a fraction thereof, and/or a cell lysate thereof, and/or fermentate thereof, and/or metabolite thereof.
29. The method of embodiment 28, wherein the first and second cosmetic active are administered to the subject's skin or scalp.
30. The method of embodiment 28, wherein the first cosmetic active agent and the at least one second cosmetic active agent are formulated in a single composition.
31. The method of embodiment 28, wherein the first cosmetic active agent and the at least one second cosmetic active agent are formulated in separate compositions.
32. The method of embodiment 28, wherein the at least one second cosmetic active agent comprises at least one antidandruff active agent.
33. The of embodiment 28, wherein the at least one second cosmetic active agent comprises at least one member selected from the group consisting of zinc pyridinethione, salicylic acid, selenium disulfide, mixed oil of thyme and catnip, octopirox and a probiotic microorganism.
34. The method of 28, wherein the at least one second cosmetic active agent comprises at least one member selected from the group consisting of keratolytic agents such as salicylic acid and sulphur in its various forms, regulators of keratinization such as zinc pyrithione, a pyridinethione salt, a trihalocarbamide, triclosan, an azole compound, an antifungal polymer, allantoin, steroids such as topical corticosteroids, tar or polytar (coal tar), undecylenic acid, fumaric acid, an allylamine and mixtures thereof, ciclopirox, octopirox, piroctone olamine, clobetasol propionate, betamethasone valerate, tea tree oil, a mixed oil of thyme and catnip, topical antifungals such as selenium sulfide, imidazole (e.g. ketoconazole), hydroxypyridones such as (e.g. ciclopirox), naturopathic agents such as Melaleuca sp. oil, Aloe vera, and a probiotic microorganism.
35. The method of embodiment 16, wherein the dandruff condition of the scalp comprises dandruff in combination with: dryness of the scalp, hyperseborrhoea of the scalp, an imbalanced ecoflora, pruritus, inflammation of the scalp, or an imbalanced barrier function of the scalp.
36. A method for preparing a cosmetic composition or dermatological composition for treating a dandruff condition, comprising combining an effective amount of at least one microorganism of the genus Yarrowia and/or a fraction thereof, and/or a cell lysate thereof, and/or fermentate thereof, and/or metabolite thereof, and/or a fraction thereof with at least one cosmetic or dermatological excipient.
37. A skin care composition for use in the treatment of a scalp disorder, comprising a fermentate of Yarrowia cells, wherein said composition reduces and/or treats said scalp disorder.
38. The skin care composition of embodiment 37, wherein all or substantially all of the Yarrowia cells have been removed from the fermentate.
39. The composition of embodiment 37, wherein the fermentate is a culture supernatant.
40. The composition of embodiment 37, further comprising one or more dermatologically or skin care acceptable component.
41. A skin care product comprising the skin care composition of any one of embodiments 37-40.
42. Fermentate of Yarrowia for use in treatment of a scalp disorder.
43. Fermentate Yarrowia for use according to embodiment 36, wherein all or substantially all of the Yarrowia cells have been removed from the fermentate.
44. Fermentate of Yarrowia for use according to embodiment 36, wherein the fermentate is a culture supernatant.
45. A skin care product comprising the fermentate of any one of embodiments 42-44.
46. A skin care composition for use in the treatment of a scalp disorder, comprising a metabolite of Yarrowia cells, wherein said metabolite is derived from the metabolism of a Yarrowia microorganism and also having efficacy in the treatment of said scalp disorder, wherein said composition reduces and/or treats said scalp disorder.
47. The skin care composition of embodiment 40, further comprising one or more dermatologically or skin care acceptable component.
48. A skin care product comprising the skin care composition of any one of embodiments 46-47.
49. A metabolite of Yarrowia for use in treatment of a scalp disorder, wherein said metabolite is derived from the metabolism of a Yarrowia microorganism and also having efficacy in the treatment of said scalp disorder.

### EXAMPLES

In the following Examples, unless otherwise stated, parts and percentages are by weight and degrees are Celsius. It should be understood that these Examples, while indicating embodiments of the disclosure, are given by way of illustration only. From the above discussion and these Examples, one skilled in the art can make various changes and modifications of the disclosure to adapt it to various usages and conditions. Such modifications are also intended to fall within the scope of the appended claims.

The following abbreviations in the specification correspond to units of measure, techniques, properties, or compounds as follows: “sec” or “s” means second(s), “min” means minute(s), “h” or “hr” means hour(s), “μL” means microliter(s), “mL” means milliliter(s), “L” means liter(s), “mM” means millimolar, “M” means molar, “mmol” means millimole(s), “ppm” means part(s) per million, “wt” means weight, “wt %” means weight percent, “g” means gram(s), “mg” means milligram(s), “μg” means microgram(s), “ng” means nanogram(s), “conc.” means concentration, “Trt” means treatment.

### Example 1

### Materials and Methods

### Yeast Strains

Yeasts were from ATCC (American Type Culture Collection), CBS (CBS-KNAW culture collection), or Phaff Yeast Culture Collection (UC Davis), Strains used are M. globosa (CBS 7966), M. furfur (CBS 1878), Y. lipolytica (ATCC 20362, ATCC 9773, ATCC 18942, ATCC 20177, CBS 2073, Phaff #50-47).

### Growth Media

Cells were grown in modified Leeming & Notman (mLN) media containing 10 g/L bacteriological peptone, 2 g/L yeast extract, 8 g/L desiccated ox bile, 10 ml/L glycerol, 0.5 g/L glycerol monostearate, 5 ml/L Tween-60, 20 ml/L olive oil or palm oil. For synthetic growth media, 6.7 g/L yeast nitrogen base without amino acids, 6 g/L dipotassium phosphate, 4 g/L monopotassium phosphate, and different amounts of lipid as carbon sources and Tween-40, −60, or −80 as emulsifier. For solid media, 15 g/L agar was added. Medium was sterilized by autoclave at 110° C. for 20 minutes.

### Lipase Activity Assay

4-Methylumbelliferyl oleate (Sigma-Aldrich, St. Louis, Mo.) was used as substrate for lipase and dissolved to 5 mg/ml in DMSO as stock solution. It was then diluted to 0.5 mg/ml in DMSO, and 50 μl was used for each reaction in 96-well plate. 100 μl filtered (0.22 micron filter) cell culture was added to each well and mixed by pipetting five times. Fluorescence intensity was measured every 10 minutes for 1 hour at 37C by the Tecan Spark® microplate reader. The excitation and emission wavelengths were 355 and 460 nm with gain of 55.

Cell counting by qPCR

Quantitative PCR or qPCR was utilized to quantitate Yarrowia lipolytica and Malassezia globosa in individual and combined culture conditions at three timepoints; Day 1, Day 4 and Day 7. The qPCR assay used for the detection of all Malassezia globosa species was as described by Clavaud et al, (2013), PLOS One, 8:10. The primers/probe used were as follows; forward primer 1: 5′ CTAAATATCGGGGAGAGACCGA (SEQ ID NO:1), and reverse primer 2: 5′ GTACTTTTAACTCTCTTTCCAAAGTGCTT (SEQ ID NO:2), and the MGB Probe 1; FAM-TTCATCTTTCCCTCACGGTAC-MGB (SEQ ID NO:3). The qPCR assay for Yarrowia lipolytica targets the SNF1 gene and includes the following primers/probe; forward primer 3: 5′ ACACCATTCCCCCCTATCTGT (SEQ ID NO:4), reverse primer 4: 5′ TGACCACCAGCATCTGTTGAA (SEQ ID NO:5) and probe 2: 5′ 6FAM-TGCCGGCGCAAAACACCTG-TAMRA. ((SEQ ID NO:6) Genomic DNA from a representative strain of both Malassezia globosa and Yarrowia lipolytica were used to generate a standard curve for absolute quantitation.

One ml (1 ml) of culture for Yarrowia lipolytica and Malassezia globosa grown individually or in combination from each time point were centrifuged to pellet the yeast. Cell pellets were then utilized for genomic DNA extraction. The DNA was extracted using the Qiagen DNeasy PowerSoil Pro DNA kit (Qiagen™ Germantown, Md.) as per the manufacturers instructions. 1.5 μl of purified genomic DNA from each sample was combined with each of the following in two separate reactions for detection of Malassezia globosa or Yarrowia lipolytica; 10 μl ABI Universal TaqMan Mix w/o UNG, 0.2 μl 100 μM Forward and Reverse Primers, 0.05 μl TaqMan Probe and 8.05 μl Molecular Biology Grade water. Each sample was run in triplicate qPCR reactions on a Quantstudio 7 Instrument as follows: 10 min 95° C.+40 cycles (95° C. 15 sec+60° C. 60 sec). Fluorescent data was collected during amplification.

Linear regression analysis was used to establish standard curves for both Malassezia globosa and Yarrowia lipolytica based on known amounts of genomic DNA (gDNA) and their corresponding qPCR derived Ct values. These standard curves were then used to determine the copy number of each organism in samples from each of the three timepoints. Average values of the triplicate qPCR reactions were reported.

### Gas Chromatography (GC) for Identification and Quantification of Lipids in the Media.

Analytical standards for oleic and palmitic acids were U46A (C18:1) and N16A (C16:0) from NuCheck Prpe, Inc. For each compound, a standard curve was obtained for subsequent quantification; standards were prepared by weight into hexane. Myristic acid (C14:0, Sigma #70079) was added to the hexane solvent as an internal standard to correct for possible deviation in the injection volumes during the GC analysis; 62 mg were added per 100 mL hexane.

Agilent Technologies 7890A gas chromatograph was used to perform the separation and quantification of free fatty acids. It was equipped with 7683B dual injector towers (front/back), a G2614A autosampler and a flame ionization detector (HD). Helium was used as a carrier gas while the fuel gas and support air to the FID was provided with VWR 26000-C034 hydrogen generator.

The analysis was carried out on an Agilent J&W DB-FFAP column, a nitroterephthalic-acid-modified polyethylene glycol (PEG) column (30 m×0.25 mm ID×0.25 um). The instrument method was as follows. The inlet was set to 250° C., with a septum purge of 3 mL/min, and 1 μL was injected with a 100:1 split mode. The temperature program was 80° C. for 2 min then 8° C./min to 250° C., holding for 10 min (post run at 50° C. for 3 min). The helium gas carrier was used at a flow rate of 1.8 mL/min, and the FID settings were 300° C., with a 40 mL/min and 450 mL/min flow for H2 and air respectively and a 41.5 mL/min make up flow.

The samples analyzed were aliquots from multiple on-going cultures of Malassezia globosa and Yarrowia lipolytica over a period of 7 days. 1 mL aliquots were taken from the cultures and frozen at −80° C. until subsequent analysis for C16:0 and C18:1 content by GC-FID. At the time of analysis, samples were allowed to thaw at ambient temperature. After 30 to 60 seconds of vigorous vortexing to homogenize each sample, 250 μL were aliquoted to 2-mL microfuge tubes. They were extracted with 250 μL of hexane; hexane spiked with C14:0 internal standard was used to check for variability in GC injections. Samples were shaken on a vortex mixer, using a microfuge tube carousel adapter for 10 min; samples were then allowed to sit at ambient temperature for 15 min before being centrifuged for 5 min at 1900× g. The hexane phase at the top, containing the extracted free fatty acids, was transferred to a glass vial for subsequent analysis by GC-FID, The present method was able to separate and quantify palmitic and oleic acids, respectively at 19.4 min and 21.6 min retention times. The accuracy of the GC analysis was verified by internal standard myristic acid, peak at 17.8 min with a 2-5% variation on the peak area throughout the assay.

### Example 2

### Growth of Malassezia Spp. and Yarrowia lipolytica in Different Lipid Media

The current example demonstrates the growth phenotypes of M. globosa and Y. lipolytica in different growth media using lipids as carbon sources. As shown in Table 1, all Yarrowia lipolytica strains grew very well on lipids tested, and M. furfur showed either good or moderate growth. However, M. globosa grew poorly in all conditions tested, especially on oleate (no growth) or olive oil (weak or no growth), and showed weak growth on palm oil or ethyl palmitate. Olive oil is mainly composed of unsaturated fatty acids (86%), especially oleic acid (78%). Palm oil is mainly composed of palmitic acid (44%) and oleic acid (40%).

Table 1 shows the growth phenotypes of yeast strains on different lipid plates. Lipids and emulsifiers used are shown on the top row. Cells were streaked on the plates and grown at 32° C. for 3 days.+++, good growth; ++, moderate growth; +, weak growth; −, no growth.

TABLE 1
Growth phenotypes of yeast strains on different lipid plates.

| | 20 ml/L | 20 ml/L | 5 ml/L | 1 ml/L ethyl |
|---|---|---|---|---|
| | Palm oil | Olive oil | Oleate | palmitate |
| Yeast used | 2 ml/L | 2 ml/L | 2 ml/L | 0.5 ml/L |
| (strain name) | Tween-40 | Tween-40 | Tween-80 | Tween-40 |
| M. globosa | + | −/+ | − | + |
| (CBS 7966) | | | | |
| M. furfur | +++ | ++ | ++ | +++ |
| (CBS 1878) | | | | |
| Y. lipolytica | +++ | +++ | +++ | +++ |
| (ATCC 20362) | | | | |
| Y. lipolytica | +++ | +++ | +++ | +++ |
| (ATCC 9773) | | | | |
| Y. lipolytica | +++ | +++ | +++ | +++ |
| (ATCC 18942) | | | | |
| Y. lipolytica | +++ | +++ | +++ | +++ |
| (ATCC 20177) | | | | |
| Y. lipolytica | +++ | +++ | +++ | +++ |
| (CBS 2073) | | | | |
| Y. lipolytica | +++ | +++ | +++ | +++ |
| (Phaff #50-47) | | | | |

Sebum, a product of the sebaceous gland, is a mixture of lipids, mainly of triglycerides, free fatty acids, wax ester, and squalene. Among fatty acids, main components of sebum lipids are sapienic add and palmitic acid, each accounting for about 30%. Oleic acid is a minor component with about 2-5% (Akaza, et al. (2014), J. Dermatology 41: 1069).

The results shown in Table 1 indicate that M. globosa was unable to utilize (and thus unable or weak growth on) Oleate (Oleic acid) and was able to grow slightly on palmitate.

In contrast, all Yarrrowia lipolitca strains ere able to grow on Oleate and Palmitate.

### Example 3

### Lipase Activities of Malassezia Spp. and Yarrowia lipolytica in Different Lipid Media

To measure secreted lipase activities, cell culture aliquots (1 ml) were collected, and cells were removed by 0.22 micron filter. 4-Methylumbelliferyl oleate (Sigma-Aldrich, St. Louis, Mo.) was used as substrate for lipase(s) in the filtered culture supernatant, and activity was measured by fluorescence.

Table 2 shows the lipase activities from cell culture supernatant of M. globosa and Y. lipolytica, cells were grown in synthetic medium containing 1 ml/L ethyl palmitate and 0.5 ml/L Tween-40 at 32° C. with shaking for 2 days (Y. lipolytica) or 5 days (M. globosa). The activities shown were from fluorescence generated by 4-methylumbelliferyl oleate hydrolysis by lipase activity. No cell (medium) is the control showing background signal.

TABLE 2
Lipase activities from cell culture supernatant of
M. globosa and Y. lipolytica.

| | Time (minutes) | | | | | | |
|---|---|---|---|---|---|---|---|
| | 0 | 10 | 20 | 30 | 40 | 50 | 60 |
| M. globosa | 0 | 821 | 1784 | 2380 | 2809 | 3085 | 3315 |
| (CBS 7966) | | | | | | | |
| Y. lipolytica | 0 | 0 | 0 | 0 | 0 | 0 | 0 |
| (ATCC 20362) | | | | | | | |
| Y. lipolytica | 0 | 0 | 0 | 0 | 0 | 0 | 0 |
| (CBS 2073) | | | | | | | |
| Y. lipolytica | 0 | 188 | 521 | 784 | 967 | 1077 | 1186 |
| (Phaff #50-47) | | | | | | | |

As shown in Table 2, M. globosa expressed the highest lipase activity in the cell free medium (secreted). On the other hand, Y. lipolytica strains ATCC 20362 and CBS 2073 did not show significant lipase activities in the cell free supernatant, suggesting their lipase activities were mostly cell-bound or cell-associated. Y. lipolytica strain Phaff #50-47 produced some lipase activity in the culture supernatant.

### Example 4

### Competitive Growth Assay on M. globosa and Y. lipolytica

The current example describes biomass changes over time when M. globosa and Y. lipolytica were grown together or separately. M. globosa needs to have lipids in the media to grow because it lacks fatty acid synthase, and the addition of lipids and emulsifier in the media generates micelles or lipid droplets which make it difficult to monitor cell growth by optical density. M. globosa also grows as cell clumps in liquid media, and therefore counting colonies after spreading a culture aliquot on the plate is not an accurate way of measuring cell growth. For more accurate cell growth measurements, qPCR method has been used to monitor cell growth by DNA content.

Table 3 shows the result of the growth competition between M. globosa and Y. lipolytica by growing the yeasts in combination or by itself using a modified Leeming & Notman medium containing palm oil. Culture aliquots were taken at 3 different time-points over 7 days, and the yeast growth was measured by qPCR. The result showed that the growth of M. globosa was affected/reduced by more than 10-fold when the two yeasts were grown together, compared to when M. globosa was grown alone. However, the growth of Y. lipolytica was virtually not affected by the presence of M. globosa, when compared to Y. lipolytica growth by itself.

Table 3 shows the qPCR based cell counts of M. globosa and Y. lipolytica (ATCC 20362) when the two yeasts were grown together or separately. M. globosa culture grown in 100 ml mLN media containing 20 ml/L palm oil for 7 days was divided into two 50 ml cultures, and one 50 ml was added with Y. lipolytica to an OD₆₀₀ of 0.1 (co-culture) and the other 50 ml was not (M. globosa alone). ‘Y. lipoytica alone’ culture was 50 ml fresh mLN medium inoculated with Y. lipolytica to an OD₆₀₀ of 0.1. Genomic DNA was prepared from 1 ml aliquot of each cell culture and used for qPCR analyses.

TABLE 3
qPCR based cell counts of M. globosa and
Y. lipolytica (ATCC 20362) when the
two yeasts were grown together or separately.

| | Day 1 | Day 4 | Day 7 |
|---|---|---|---|
| M. globosa alone | 2.62 × 10⁵ | 2.44 × 10⁵ | 1.50 × 10⁶ |
| Y. lipolytica alone | 6.60 × 10³ | 1.00 × 10⁶ | 1.16 × 10⁶ |
| M. globosa in co- | 5.10 × 10⁵ | 2.44 × 10⁵ | 1.16 × 10⁵ |
| culture | | | |
| Y. lipolytica in co- | 7.95 × 10³ | 1.11 × 10⁶ | 1.34 × 10⁶ |
| culture | | | |

As shown in Table 3, surprisingly and unexpectedly the presence of Y. lipolytica in a medium comprising M. globosa, resulted in a significant reduction of growth of comprising M. globosa (e.g. Y. lipolytica is inhibiting Malassezia spp), and as such indicated that can be used a microbial treatment for a dandruff condition.

### Example 5

### GC Analysis for Y. lipolytica Lipid Consumption

The current example describes oleic and palmitic acids consumption by Y. lipolytica and M. globosa. It was to show that oleic acid free fatty acid (FFA) generated by lipase activity of M. globosa was consumed by Y. lipolytica. Gas Chromatography (GC) was used to measure the amounts of FFA in the media.

A modified Leeming & Notman (mLN) medium containing palm oil was used for cell culturing. M. globosa was grown in 100 ml mLN medium at 32° C. with shaking for 7 days, and the culture supernatant was collected by centrifugation and split into two 50 ml and transferred to 250 ml flasks. In one of the 50 ml culture supernatant, 500 μl from a Y. lipolytica overnight culture was added to a final OD₆₀₀ of 0.1. Samples (1 ml) were collected before and after centrifugation for GC analysis. Most of M. globosa had been removed by centrifugation, but not all M. globosa cells were removed from supernatant. The M. globosa supernatant cultures with or without Y. lipolytica addition were incubated at 32° C. with shaking. 1 ml aliquots were collected for GC analysis after 1, 2, 3, 4, and 7 days of incubation.

Table 4 shows the result of GC analysis. After centrifugation to remove most of M. globosa, both palmitic and oleic acids were significantly reduced. This could be because some FFA's were associated with cells and spun down together during the centrifugation. The amounts of palmitic and oleic FFA's between ‘M. globosa Supernatant’ and ‘M. globosa Supernatant+Y. lipolytica’ were comparable after centrifugation (Day 0).

In case of ‘M. globosa Supernatant’, the amounts of palmitic and oleic FFA's were largely unchanged until Day 4 since most of M. globosa cells were removed by centrifugation. However, there was clear reduction in FFA in samples after Day 7. On the other hand, when Y. lipolytica was added to ‘M. globosa Supernatant’, both palmitic and oleic FFA were quickly consumed, but oleic acid was consumed faster than palmitic acid.

Table 4 shows the free fatty acid quantification by GC. M. globosa was grown in 100 ml mLN media containing 20 ml/L palm oil for 7 days and divided into two 50 ml supernatant aliquots after centrifugation. One 50 ml aliquot was added with Y. lipolytica to an OD₆₀₀ of 0.1 (M. globosa Supernatant+Y. lipolytica), and the other 50 ml was not added with Y. lipolytica (M. globosa Supernatant). Samples were taken during incubation at 32° C. with shaking as indicated in the table, and analyzed on palmitic and oleic FFA's. Relative amounts of FFA compared to the Day 0 sample are shown in parenthesis.

TABLE 4
Free fatty acid quantification by GC of M. globosa Supernatant
and M. globosa Supernatant + Yarrowia lipolytica

| | | | M. globosa Supernatant + | |
|---|---|---|---|---|
| | M. globosa Supernatant | | Y. lipolytica | |
| | Palmitic FFA, | Oleic FFA, | Palmitic FFA, | Oleic FFA, |
| | g/L | g/L | g/L | g/L |
| | (% Day 0) | (% Day 0) | (% Day 0) | (% Day 0) |
| Before | 4.52 | 3.05 | 4.52 | 3.05 |
| centrifuge | | | | |
| After | 2.4 (100%) | 1.92 (100%) | 2.1 (100%) | 1.76 (100%) |
| centrifuge: | | | | |
| Day 0 | | | | |
| Day 1 | 2.25 (93%) | 1.78 (93%) | 2.05 (97%) | 1.58 (90%) |
| Day 2 | 1.98 (82%) | 1.43 (74%) | 1.89 (90%) | 1.27 (72%) |
| Day 3 | 2.15 (89%) | 1.53 (80%) | 1.51 (72%) | 0.65 (37%) |
| Day 4 | 1.84 (76%) | 1.36 (71%) | 1.12 (53%) | 0.27 (16%) |
| Day 7 | 0.95 (39%) | 1.09 (57%) | 0.04 (2%) | 0.04 (2%) |

Surprisingly and unexpectedly, the results in Table 3 (Example 4) showed that when Y. lipolytica and M. globosa were grown together, growth of M. globosa in presence of Y. lipolytica was significantly reduced (e.g. Y. lipolytica is inhibiting Malassezia spp), and as such indicated that microorganisms of the genus Yarrowia can be used as a microbial treatment for a dandruff condition. As shown in Table 2, M. globosa expressed lipase activity in the cell free medium (indicating that the lipase was secreted), but Y. lipolytica did not show significant lipase activities in the cell free supernatant, presumably because Yarrowia's lipase activities were mostly cell-bound or cell-associated. This is a surprising and favorable feature as a microbial treatment because the lipase activities would not be left on the skin and cause free fatty acid accumulation after microbial treatment is completed. The result in Table 4 also showed that oleic free fatty acid (FFA) generated by lipase activity of M. globosa was efficiently consumed by Y. lipolytica but not by M. globosa, which is also in agreement with the result (Table 1) showing that M. globosa was unable to utilize (and thus unable or weak growth on) oleic acid but efficiently assimilated by Yarrowia lipolytica. Since oleic free fatty acid (FFA) is considered proinflammatory, efficient removal of this FFA is an important attribute of the Yarrowia microbial treatment against dandruff or other skin disorders caused by proinflammatory oleic FAA.

### Example 6

### Flow Cytometry Analysis of M. globosa Growth Inhibition by Y. lipolytica Cell Free Culture Supernatant (Cell Free Fermentate)

Malassezia species (Malassezia spp.) implicated in skin disorders do not have fatty acid synthase for lipid synthesis and therefore rely on sebum lipids from host for their growth (Xu et al., (2007) PNAS, 104: 18730). The growth medium for Malassezia species was a modified Leeming & Notman (mLN) media that contained 10 g/L bacteriological peptone, 2 g/L yeast extract, 8 g/L desiccated ox bile, 10 ml/L glycerol, 0.5 g/L glycerol monostearate, 5 ml/L Tween-60 and 20 ml/L palm oil.

Lipid emulsion by the presence of lipids and surfactants in the mLN media as well as cell clumping phenotype of Malassezia spp, make it difficult to monitor cell growth by optical density measurement or colony counting after spreading a culture aliquot on plates, To circumvent this issue, flow cytometry with cell staining dyes was used to quantitatively measure cell growth in lipid emulsion media. The flow cytometry analysis was conducted as follows to monitor the growth of M. globosa when challenged with Y. lipolytica cell free culture supernatants.

First, to generate different types of fermentates from Yarrowia lipolytica (Y. lipolytica) cultures, Y. lipolytica ATCC 20362 strain was grown in YPD (10 g/L yeast extract, 20 g/L peptone, 20 g/L glucose), YPG (10 g/L yeast extract, 20 g/L peptone, 20 ml/L glycerol), mLN with 20 ml/L palm oil, or mLN with 20 ml/L olive oil media for 5 days with initial OD₆₀₀ of 0.1 at 32° C. with shaking at 250 rpm. Cell free Yarrowia culture supernatant (also referred to as cell free fermentate) was prepared by filtering the Yarrowia cell culture though 0.22 μm filter membrane twice to remove cells and stored frozen at −20° C.

To monitor Malassezia globosa (M. globosa) growth inhibition by Y. lipolytica cell free fermentate, three-day M. globosa culture inoculum was diluted 10 times in fresh mLN media containing 20 ml/L palm oil. The diluted culture was then further diluted 5 times in 0.2 M sodium phosphate buffer (pH 6.0). Two hundred (200) μl of Y. lipolytica cell free culture supernatant or media alone was added to 800 μl of the diluted M. globosa culture in 96-deep well plates (Biotix, San Diego, Calif.) and incubated at 32° C., shaken at 350 rpm with constant 85% humidity.

At different time points, 50 μl aliquot from each culture was subjected to mild sonication with amplitude of 5 and 1 sec on/off cycle for 30 sec using Qsonica 0700 (Qsonica, Newtown, Conn.). 20 μl of the sonicated cell culture was mixed with 50 μl phosphate-buffered saline, pH 7.4 (PBS), 10 μl diluted (1:1,000 in PBS) Cyto BC Green (Invitrogen), 10 μl diluted (1:1,000 in PBS) propidium iodide (Invitrogen), and 10 μl of diluted (1:200 in PBS) Concanavalin A (Invitrogen), and incubated for 20 min at room temperature. The samples were analyzed by flow cytometry using a Novocyte Quanteon (Asea). SytoBC, a cell permeable DNA/RNA intercalating dye was excited by the 488 laser and detected using a 530/30 bandwidth filter. Concanavalin A, a mannose binding lectin labeled with alexaFluor 640, was excited with the 637 nm laser and detected using a 660/20 bandwidth filter. Events with high mannose and high nucleic acid content were determined to be Malassezia cells versus events triggered by emulsion droplets which had comparatively low signal intensity in these channels. Malassezia cell counts were determined by gating on high sytoBC, high ConA events and recording the event/ul values for each sample.

Table 5 shows the flow cytometric analysis of M. globosa culture after the addition of various cell free culture supernatants from Y. lipolytica ATCC 20362 or antifungal agent amphotericin B (2.5 μg/ml), Percentages of M. globosa growth inhibition compared to respective media controls are shown in Table 5. The values are average of duplicate samples.

TABLE 5
Flow cytometric analysis of M. globosa culture after the
addition of various cell free culture supernatants from
Y. lipolytica ATCC 20362 or antifungal agent
amphotericin B (2.5 μg/ml).

| | Day 1 | Day 3 |
|---|---|---|
| | (% M. globosa | (% M. globosa |
| | growth inhibition) | growth inhibition) |
| Amphotericin B | 4% | 88% |
| Yarrowia YPD cell free | 67% | 81% |
| fermentate | | |
| Yarrowia YPG cell free | 0% | 73% |
| fermentate | | |
| Yarrowia mLN with olive | 22% | 60% |
| oil cell free fermentate | | |
| Yarrowia mLN with palm | 0% | 35% |
| oil cell free fermentate | | |

For all Yarrowia fermentates tested, M. globosa growth was reduced by the addition of Y. lipolytica cell free culture supernatants over time, compared to that of respective media alone controls. Among the Y. lipolytica cell free culture supernatants, YPD fermentate had the largest effect on M. globosa growth after 3-day incubation, which showed 81% reduction in cell counts compared to media control.

### Example 7

Flow Cytometry Analysis of M. globosa Growth Inhibition by Cell Free Culture Supernatant from 2 Different Y. lipolytica Strains

Flow cytometric analysis method was used to monitor the growth of M. globosa when challenged with Y. lipolytica cell free culture supernatants. Y. lipolytica strains ATCC 20362 or ATCC 9773 was grown in YPD (10 g/L yeast extract, 20 g/L peptone, 20 g/L glucose), YPG (10 g/L yeast extract, 20 g/L peptone, 20 ml/L glycerol), mLN (10 g/L bacteriological peptone, 2 g/L yeast extract, 8 g/L desiccated ox bile, 10 ml/L glycerol, 0.5 g/L glycerol monostearate, 5 ml/L Tween-60) with 20 ml/L palm oil, or mLN with 20 ml/L olive oil for 5 days with initial OD₆₀₀ of a 1, at 32° C. with shaking at 250 rpm. Cell free Yarrowia culture supernatant (also referred to as cell free fermentate) was prepared by filtering the Yarrowia cell culture though 0.22 μm filter membrane twice to remove cells and stored at −20° C.

M. globosa culture inoculum was diluted 10 times in fresh mLN medium containing 20 ml/L palm oil. The diluted culture was then further diluted 5 times in 0.2M sodium phosphate buffer (pH 6.0). Two hundred (200) μl of Y. lipolytica cell free culture supernatant or media alone was added to 800 μl of the diluted M. globosa culture in 96-deep well plates (Biotix, San Diego, Calif.) and incubated at 32° C. with shaking at 350 rpm with constant 85% humidity. After 3 days of incubation, 50 μl aliquot from each culture was analyzed by flow cytometer using cell staining dyes, as described in Example 6.

Percentage of M. globosa growth inhibition compared to respective media controls are shown in Table 6. The values are average of duplicate samples.

TABLE 6
Flow cytometric analysis of M. globosa culture 3 days
after the addition of various cell free culture supernatants
from Y. lipolytica ATCC 20362 or ATCC 9773.

| | % M. globosa growth inhibition on day3 | |
|---|---|---|
| Y. lipolytica culture | ATCC 20362 | ATCC 9773 |
| Yarrowia YPD cell free | 65% | 69% |
| fermentate | | |
| Yarrowia YPG cell free | 46% | 61% |
| fermentate | | |
| Yarrowia mLN with olive | 41% | 58% |
| oil cell free fermentate | | |
| Yarrowia mLN with palm | 59% | 42% |
| oil cell free fermentate | | |

M. globosa growth was reduced by the addition of Y. lipolytica cell free fermentate (cell free culture supernatant), compared to respective media alone controls. Among the Y. lipolytica cell free culture supernatants from ATCC 20362 and ATCC 9773, YPD fermentate had the largest effect on M. globosa growth after 3-day incubation, which showed about 65-69% reduction in M. globosa, compared to media controls.

### Example 8

### Removal of Biofilm of Malassezia Species by Yarrowia Fermentate

Skin is a unique environment where microbes often exist as biofilms (Brandwein, et al., NPJ Biofilms Microbiomes 2:3, 2016). The biofilms can form on the epithelial surfaces of the skin or inside the follicles. In addition to cells, a biofilm consists of extracellular components such as exopolysaccharides, proteins, and DNA. This complex structure can be a physical and chemical barrier for certain compounds. But more importantly, the physiology of the microbes in the state of biofilm is very different than those in planktonic state. This is especially true for their ability to counter environmental stress and to resist various antimicrobial treatments, which provides remarkable therapeutic challenges (Koo, et al., Nature Reviews Microbiology 15:740-755, 2017).

Yeast Malassezia species isolated from both healthy and unhealthy skin have been shown to form biofilms in vitro (Angiolella, et al., Med Mycol. 0:1-7, 2020). These isolates of Malassezia globosa(M. globosa) can be highly adherent and/or hydrophobic as well as biofilm producers. Malassezia species in the form of biofilm have been shown to have a significant decrease in their susceptibility to antifungal agents (Figueredo, et al., Medical Mycology 8:863-867, 2013; Bumroogthai, et al., Medical Mycology 54:544-549, 2016). Biofilm adherence and hydrophobicity was suggested as virulence factor for Malassezia (Allen, et al., J. of Clinical & Experimental Dermatology Research 6:311, 2015; Angiolella, et al., Medical Mycology 56:110-116, 2018). Thus, strategies to remove Malassezia biofilm can be beneficial to treat various skin conditions caused by this group of organisms.

In this example, M. globosa ATCC MYA-4612 was used for the development of a biofilm assay as described below

M. globosa ATCC IMA-4612 was grown in mLN media with 20 ml/L palm oil (described in Example 6) at 32° C. in a rotary shaker with a speed of 100 rpm. After 3 days of incubation, 25 μl culture was inoculated into wells of a polystyrene 96 well plate with 150 μl of mLN media using palm oil as the carbon source. The plate was incubated at 32° C. without shaking for 48 hours to allow growth of M. globosa as both suspending cells (planktonic cells) and as sessile biofilm cells attached to the wall of wells in the microtiter plate.

After the biofilm was formed, the growth medium and the unattached cells were removed, and wells were washed once with 1× phosphate buffered saline solution (PBS). PBS is a pH-adjusted blend of ultrapure-grade phosphate buffers and saline solutions which, when diluted to a 1× working concentration, contains 137 mM NaCl, 2.7 mM KCl, 8 mM Na₂HPO₄, and 2 mM KH₂PO₄. After washed, 250 μl cell free supernatant (cell free fermentate) of two different strains of Yarrowia lipolytica (ATCC20362 and ATCC9773) was added to evaluate their efficacy for biofilm removal. 250 ul of 1× PBS was also added as a control.

As described in Example 7, Y. lipolytica strains ATCC 20362 or ATCC 9773 were grown in YPD (10 g/L yeast extract, 20 g/L peptone, 20 g/L glucose), YPG (10 g/L yeast extract, 20 g/L peptone. 20 ml/L glycerol), mLN (10 g/L bacteriological peptone, 2 g/L yeast extract, 8 g/L desiccated ox bile, 10 mi/L glycerol, 0.5 g/L glycerol monostearate, 5 ml/L Tween-60) with 20 ml/L palm oil, or mLN with 20 ml/L olive oil for 5 days with initial OD₆₀₀ of 0.1, at 32° C. with shaking at 250 rpm. Cell free Yarrowia culture supernatant (also referred to as cell free fermentate) was prepared by filtering the Yarrowia cell culture though 0.22 μm filter membrane twice to remove cells and stored at −20° C.

In this example, Yarrowia fermentate was generated using mLN medium with 20 ml/L olive oil. After the addition of Yarrowia fermentate or 1×PBS, the biofilm plate was incubated at 32° C. for 15 min without shaking for the biofilm removal reaction. After incubation, the supernatant or PBS was removed. The amount of biofilm remaining in the wells were quantified after staining. The staining of biofilm was carried out by adding 250 μl of 0.1% crystal violet dissolved in water. The plate was incubated for 3 min at room temperature. After staining, 250 μl of 1× PBS was added to each well to remove the unbound dye. This process was repeated 1 more time. After wash, 250 μl of 70% ethanol was added to each well and the plate incubated for 5 min at room temperature to release the dye. The intensity of the dye was measured at 570 nm with a microtiter plate reader. The OD reading was used to quantify the amount of biofilm remained after each treatment. The wells treated with PBS were used as a reference to calculate the percentage of biofilm removal.

The results of the removal of Malassezia biofilm formation with culture supernatant (fermentate) from Yarrowia strains is shown in Table 8.

TABLE 7
Percent biofilm removal of Malassezia biofilm formation
with culture supernatant (fermentate) from Yarrowia strains.

| | | Biofilm |
|---|---|---|
| Treatment | OD (570 nm) | removal (%) |
| PBS | 1.81 | 0 |
| ATCC 20362 | 0.88 | 52 |
| ATCC 9773 | 0.50 | 73 |

Addition of cell free fermentate (supernatant) from Yarrowia lipolytica strain ATCC 20362 led to a biofilm reduction by 52% as compared to the PBS medium control, while addition of supernatant from strain ATCC 9773 resulted in 73% reduction as compared to the PBS medium control. This experiment demonstrated the efficacy of cell free fermentates from two different Yarrowia strains for removal of Malassezia biofilm.

### Example 9

### Prevention and Reduction of Biofilm Formation of Malassezia Species by Yarrowia Fermentate

As stated previously, microbiome on skins surfaces including scalp is primarily present as biofilm communities. Just as important as the removal of pre-formed pathogenic biofilm as described in Example 8, prevention and reduction of biofilm growth for Malassezia species is another strategy for the treatment of seborrhoeic dermatitis. In this example, the ability of Yarrowia fermentate to prevent the formation of Malassezia biofilm formation was evaluated.

Malassezia globosa (M. globosa) ATCC MYA-4612 was used for biofilm growth assay. As described in Example 6, the strain was grown for 3 days in mLN media with 20 ml/L palm oil at 32° C. in a rotary shaker with a speed of 100 rpm as the starting culture. In a typical biofilm growth assay, 10 μl culture was inoculated into wells of a polystyrene 96 well plate containing 90 μl solution. This solution was consisted of mLN media with 20 ml/L palm oil with or without Yarrowia fermentate. The final volume was 100 μl. The palm oil concentration was 20 ml/L.

As described in Example 7, Y. lipolytica strains ATCC 20362 or ATCC 9773 was grown in YPD (10 g/L yeast extract, 20 g/L peptone, 20 g/L glucose), YPG (10 g/L yeast extract, 20 g/L peptone, 20 ml/L glycerol), mLN (10 g/L bacteriological peptone, 2 g/L yeast extract, 8 g/L desiccated ox bile, 10 ml/L glycerol, 0.5 g/L glycerol monostearate, 5 ml/′L Tween-60) with 20 ml/L palm oil, or mLN with 20 ml/L olive oil for 5 days with initial OD₆₀₀ of 0.1, at 32° C. with shaking at 250 rpm. Cell free Yarrowia culture supernatant (also referred to as cell free fermentate) was prepared by filtering the Yarrowia cell culture though 0.22 μm filter membrane twice to remove cells and stored at −20° C.

In this example, Yarrowia fermentate was generated using YPD. Different concentrations of fermentate (5, 10, and 20 μl) was used for the reduction assay. As controls, an equal concentration of YPD media (5, 10, and 20 μl) was used. The biofilm assay plate was incubated at 32° C. without shaking for 48 hours to allow biofilm growth of M. globosa on the wall of wells in the microtiter plate.

After the biofilm was formed, the growth medium and the unattached cells were removed, and wells were washed once with 1× phosphate buffered saline solution (PBS). PBS is a pH-adjusted blend of phosphate buffers and saline solutions which, when diluted to a 1× working concentration, contains 137 mM NaCl, 2.7 mM KCl, 8 mM Na₂HPO₄, and 2 mM KH₂PO₄. The amount of biofilm remaining in the wells were quantified after staining with the addition of 150 μl of 0.1% crystal violet dissolved in water. The plate was incubated for 3 min at room temperature after the addition of the dye. After staining, 150 μl of 1× PBS was added to each well to remove the unbound dye. This process was repeated 1 more time. After wash, 150 μl of 70% ethanol was added to each well and the plate incubated for 5 min at room temperature to release the dye. The intensity of the dye was measured at 570 nm with a microtiter plate reader. The OD reading was used to quantify the amount of biofilm remained after each treatment. The wells treated with YPD medium control were used as a reference to calculate the percentage of biofilm removal by Yarrowia supernatant.

The results of the Malassezia biofilm formation with culture supernatant (fermentate) from Yarrowia strains is shown in Table 9.

TABLE 9
Percent Reduction of biofilm formation by M. globosa
by different amount of Yarrowia fermentate.

| | ATCC 9733 | ATCC 20362 |
|---|---|---|
| Volume | (%) | (%) |
| 5 μl | 35.6 | 35.3 |
| 10 μl | 57.3 | 34.8 |
| 20 μl | 87.0 | 88.0 |

Addition of 5 μl supernatant from Yarrowia lipolytica strains inhibited the biofilm formation of Malassezia globosa by about 35%. Addition of 20 μl of supernatant inhibited the biofilm formation by over 80%. This experiment demonstrated the potential efficacy of supernatant from Yarrowia strains for treatment of Malassezia associated conditions.

That what is claimed:

1. A skin care composition for use in the treatment of a scalp disorder, comprising an effective amount of at least one microorganism of the genus Yarrowia and/or a fraction thereof, and/or a cell lysate thereof, and/or fermentate thereof, and/or metabolite thereof, wherein said composition reduces and/or treats said scalp disorder.

2. The skin care composition of claim 1, wherein the scalp disorder is selected from the group consisting of a dandruff condition of the scalp (seborrheic dermatitis), unbalanced ecoflora of the scalp, discomfort of the scalp, tinea versicolor, dry skin, irritated skin, or any one combination thereof.

3. The skin care composition of claim 1, further comprising one or more antidandruff active agents.

4. The skin care composition of claim 1, wherein the composition degrades lipids selected from the group consisting of palmtic acid, oleic acid, and any one combination thereof.

5. The skin care composition of claim 1, wherein the composition reduces the growth of Malassezia species.

6. The skin care composition of claim 1, wherein the composition removes biofilm of Malassezia species.

7. The skin care composition of claim 1, wherein the composition prevents or reduces biofilm formation of Malassezia species.

8. The skin care composition of claim 1, further comprising at least one additional compound selected from the group consisting of an excipient, a preservative, a pH adjuster.

9. The skin care composition of claim 1, comprising at least one microorganism of the genus Yarrowia and/or a fraction thereof, and/or a cell lysate thereof, and/or fermentate thereof, and/or metabolite thereof, selected from the group consisting of Yarrowia lipolytica ATCC 20362, Yarrowia lipolytica ATCC 9773, Yarrowia lipolytica ATCC 18942, Yarrowia lipolytica ATCC 20177, Yarrowia lipolytica CBS2073, Yarrowia lipolytica Phaff #50-47, or any one combination thereof.

10. Use of an effective amount of the skin care composition of any preceding claim in a skin care product.

11. A skin care product comprising an effective amount of the skin care composition of any one of claim 1-9 and one or more dermatologically or skin care acceptable component.

12. The skin care product of claim 11, wherein said effective amount of the skin care composition is at least about 1%, 2%, 3%, 4% up to 5% on a weight basis relative to a total weight of said skin care product.

13. A method for treating a scalp disorder in a subject in need thereof, comprising administering an effective amount of at least one microorganism of the genus Yarrowia and/or a fraction thereof, and/or a cell lysate thereof, and/or fermentate thereof, and/or metabolite thereof, to said subject.

14. The method according to claim 13, wherein the scalp disorder is selected from the group consisting of a dandruff condition of the scalp (seborrheic dermatitis), unbalanced ecoflora of the scalp, discomfort of the scalp, tinea versicolor, dry skin, irritated skin, or any one combination thereof.

15. The method according to claim 13, wherein the microorganism and/or a fraction thereof, and/or a cell lysate thereof, and/or fermentate thereof, and/or metabolite thereof is administered topically.

16. A method for treating and/or reducing a dandruff condition of the scalp in a subject in need thereof, comprising administering an effective amount of at least one microorganism of the genus Yarrowia and/or a fraction thereof, and/or a cell lysate thereof, and/or fermentate thereof, and/or metabolite thereof, to said subject.

17. The method according to claim 16, wherein the microorganism and/or, the fraction thereof, and/or the cell lysate thereof, and/or metabolite thereof, is administered topically.

18. The method of claim 16, wherein the at least one microorganism of the genus Yarrowia and/or a fraction thereof, and/or a cell lysate thereof, and/or fermentate thereof, and/or metabolite thereof is formulated in a single composition.

19. The method of claim 16, wherein the composition is administered to the subject's skin or scalp.

20. The method of claim 16, wherein the at least one microorganism of the genus Yarrowia and/or a fraction thereof, and/or a cell lysate thereof, and/or fermentate thereof, and/or metabolite thereof is selected from the group consisting of Yarrowia lipolytica ATCC 20362, Yarrowia lipolytica ATCC 9773, Yarrowia lipolytica ATCC 18942, Yarrowia lipolytica ATCC 20177, Yarrowia lipolytica CBS2073, Yarrowia lipolytica Phaff #50-47, or any one combination thereof.

21. The method of claim 16, wherein the at least one microorganism of the genus Yarrowia and/or a fraction thereof, and/or a cell lysate thereof, and/or fermentate thereof, and/or metabolite thereof, reduces the growth of Malassezia species.

22. The method of claim 16, wherein the at least one microorganism of the genus Yarrowia and/or a fraction thereof, and/or a cell lysate thereof, and/or fermentate thereof, and/or metabolite thereof removes biofilm of Malassezia species.

23. The method of claim 16, wherein the at least one microorganism of the genus Yarrowia and/or a fraction thereof, and/or a cell lysate thereof, and/or fermentate thereof, and/or metabolite thereof prevents or reduces biofilm formation of Malassezia species.

24. The method of claim 16, wherein the at least one microorganism of the genus Yarrowia and/or a fraction thereof, and/or a cell lysate thereof, and/or fermentate thereof, and/or metabolite thereof degrades lipids selected from the group consisting of palmitic acid, oleic acid, and any one combination thereof.

25. The method of any one of claims 16-24, wherein the effective amount of at least one microorganism of the genus Yarrowia and/or a fraction thereof, and/or a cell lysate thereof, and/or fermentate thereof, and/or metabolite thereof is administered as a skin care composition or a skin care product.

26. A method for treating and/or reducing a dandruff condition of the scalp in a subject in need thereof, comprising topically administering a skin care product comprising a skin care composition of any one of claim 1-9 to said subject.

27. The method of claim 26, wherein the skin care product is a selected from the group consisting of a lotion, a serum, a jelly, a cream, a gel, an emulsion, a solid cosmetic, a mask, a patch, and a stick comprising at least 1%, 2%, 3%, 4% up to 5% of said skin care composition on a weight basis relative to a total weight of said skin care product.
